(19) United States
(12) Patent Application Publication
(10) Pub. No.: US 2010/0297749 A1
(43) Pub. Date: Nov. 25, 2010

(54) METHODS AND SYSTEMS FOR BIOFUEL PRODUCTION

(75) Inventors: ALEX M. ARAVANIS, San Diego, CA (US); Brian L. Goodall, Spring, TX (US); Michael Mendez, San Diego, CA (US); Jason L. Pyle, San Diego, CA (US); Jaime E. Moreno, Laguna Nigel, CA (US)

Correspondence Address: Sapphire Energy, Inc., 3115 Merryfield Row, San Diego, CA, 92121, US

(73) Assignee: SAPPHIRE ENERGY, INC., SAN DIEGO, CA (US)

(21) Appl. No.: 12/763,068

(22) Filed: Apr. 19, 2010

Related U.S. Application Data
(60) Provisional application No. 61/171,404, filed on Apr. 21, 2009.

(51) Int. Cl. C12M 1/00

(52) U.S. Cl. 435/289.1

(57) ABSTRACT

The present disclosure relates to methods and systems for biofuel production. Systems of integrated biorefineries (IBR) and methods of using IBRs for producing fuel compositions and other products are provided herein. The IBRs can use algae for generating biofeedstock to produce the fuel compositions.

### CROSS REFERENCE TO RELATED APPLICATIONS

This application claims the benefit of U.S. Provisional Patent Application Ser. No. 61/171,404, filed Apr. 21, 2009, which is herein incorporated by reference in its entirety for all purposes.

### BACKGROUND

Carbon-based fossil fuels, such as coal, petroleum and natural gas, are finite and non-renewable resources. At the current consumption rate, supplies of fossil fuels will be exhausted in the foreseeable future. Burning fossil fuels has resulted in a rise in the concentration of carbon dioxide (CO₂) in the atmosphere, which is believed to have caused global climate change.

Biofuels are viable alternatives to fossil fuels for several reasons. Biofuels are typically renewable energy sources produced from biomass, a material derived from recently living organisms. Because transportation-related gasoline consumption represents the majority of all liquid fossil fuel use, supplementing or replacing gasoline with liquid biofuels is expected to reduce our reliance on fossil fuels and carbon dioxide production.

The present invention provides methods and systems for producing biofuels that can aid in abating the rise in CO₂, while making use of resources that are typically not useful, such as non-arable land. The systems can be integrated and self-sustaining, while generating a net output of biofuels.

### SUMMARY

Disclosed herein are systems for generating biofuels that can be integrated and are referred to herein as Integrated Biorefineries (IBRs). An IBR has various elements with inputs and outputs that are interconnected, such that a resulting product or byproduct from one unit is inputted to another unit.

The IBR comprises a production area, or growth/production unit, for growing an organism that produces an oil composition and a refinery that converts the oil composition to gasoline, diesel, jet fuel or some combination thereof. The IBR can further comprise a hydrogen source, which can supply the H₂ through a pipeline. In some embodiments, the IBR further comprises a second refinery. Both refineries can perform cracking, transesterification, hydroprocessing, or isomerization. For example, the hydroprocessing can be hydrotreating, hydrocracking, or hydroisomerization. The refinery can perform hydrodenitrogenation (HDN), hydrodeoxygenation (HDO), or hydrodemetallization (HDM).

In some embodiments, the first refinery produces jet fuel and diesel, while the second refinery produces gasoline. Furthermore, the two refineries can be in close proximity, or adjacent to one other, for example, within 5, 10, 15, 20, 30, 40, 50, or 100 miles within each other. The second refinery can also produce H₂, light hydrocarbons and naphtha that is transported to the first refinery. The second refinery can also produce CO₂ from which is transported to the production area. The second refinery; or another unit of the IBR, can also be a source of flue gas that produces at least 150,000 MT/yr of CO₂ to the production area.

The IBRs can also comprise a processing unit, such that the processing unit extracts an oil composition from the organism. For example, the processing unit can perform one or more of the following steps: degumming, bleaching, deodorizing, solid-liquid extraction using hexane. The processing unit can also separate solid components or extracts from the organism. The solid components, or solid extracts can comprise cell walls or cellulose. The IBR can also further comprise a conduit for delivering water or salt from said processing unit to the production area. The IBR can further comprise another processing unit, or waste processing unit, for processing the non-oil components, such as the solid extracts. For example, the non-oil components, such as the solid extracts may be processed by anaerobic digestion, aerobic digestion or used for feedstuffs. In some embodiments, the IBR comprises a conduit for delivering nutrients and CO₂ from the waste processing unit, or processing unit for non-oil components, to the production unit.

In some embodiments, the IBR comprises a refining unit for hydrotreating and a second refining unit that is a catalytic cracking unit. The light hydrocarbons and naphthas produced by the hydrotreating unit can be delivered to the catalytic cracking unit, while H₂ from the catalytic cracking unit is delivered to the hydrotreating unit.

In other embodiments, the IBR comprises an open pond comprising algae, which may be genetically modified, and a refinery for converting an oil composition from the algae to one or more fuel products, and the IBR can produce at least 300 bpd of green diesel. In other embodiments, the IBR comprises a production unit for growing an organism; a processing unit for extracting an oil composition from said organism; a refinery for refining said oil composition to produce jet fuel, diesel, and/or gasoline; a waste processing unit for processing residual matter; and, a conduit for delivering a byproduct from said waste processing unit to said production unit that is used for growth or maintenance of the organism. The byproducts can comprise carbon dioxide, hydrogen, or minerals. Also disclosed herein is an IBR that converts fatty acids to diesel and/or jet fuel. The IBR comprises a production area for growing an organism; an extracting unit for extracting an oil product comprising fatty acids or triglycerides from the organism; a processing unit for performing transesterification of said fatty acids or triglycerides; and, a first refining unit for refining the transesterified fatty acids or triglycerides into diesel or jet fuel. The production area, extracting unit, processing unit and first refining unit of the IBR can be in close proximity to one another (e.g. within 5, 10, 15, 20, 30, 40, 50, or 100 miles within each other).

Also disclosed herein are methods of using the IBRs disclosed herein for producing jet fuel, diesel fuel, and gasoline. Methods for producing products used for animal feed and generating power (such as methane, or other biofuels) using the IBRs are also provided. For example, a method for making jet fuel, diesel and gasoline from a single feedstock comprising growing an organism in a production field adjacent to a petroleum refinery; collecting an oil composition from the organism, performing a first refining step to produce jet fuel and diesel from the oil composition, and performing a second refining step to produce gasoline from the oil composition, is provided herein. The method can further comprise processing non-oil components from the organism to produce animal feed, biofuel, or other products to generate power.

Also provided herein is a method for making fuel comprising growing algae near a petroleum refinery, delivering CO₂ from the petroleum refinery to the algae, and refining oil from the algae in the petroleum refinery. The method can further comprise processing the non-oil compositions from the algae and delivering at least part of the processed non-oil compositions to algae to maintain growth. In some embodiments, the method for producing a fuel composition comprises developing a microorganism strain; growing the microorganism using at least 20,000 acre-ft/yr of brackish water; harvesting the microorganism; extracting from the microorganism an oil composition; and, refining the oil composition to produce a fuel composition. In yet other embodiments, the method for producing a fuel composition comprises growing a microorganism, which can be genetically modified, having one or more desired traits; harvesting the microorganism; extracting from the microorganism an oil composition; transporting the oil composition by a pipeline to a refinery; and, refilling the oil composition to produce a fuel composition. In some embodiments, the desired traits can be herbicide resistance, increased salt tolerance, ability to flocculate, or ability to produce one or more enzymes not naturally produced by the microorganism. For example, the enzyme can be in the lipid synthesis pathway or isoprenoid production pathway.

A method for producing at least approximately 80, 90, 9,000, 50,000 or 100,000 barrels per day (bpd) of one or more fuel compositions is also provided. The method can comprise growing a microorganism, which can be genetically modified; producing an oil composition from the microorganism; and refining the oil composition to produce the fuel composition. Also provided herein is a method for making diesel or jet fuel comprising: transforming an algae with a fatty acid synthase enzyme; growing said algae in an open pond system; collecting more than 3000 bpd of fatty acids or triglycerides from the algae; and, refining the fatty acids or triglycerides to make diesel or jet fuel.

In some embodiments, the IBR can produce approximately 80 barrels per day (bpd) of jet fuel and diesel using approximately 300 acres of land, 245,000 standard cubic feet per day (SCFD) of H₂, 2,500 acre-ft/yr of water, or approximately 65,000 MT/yr of CO₂. The CO₂ sequestering, or CO₂ capture of the IBR can be approximately 56 MT/day. In some embodiments, the IBR can comprise producing at least 90 bpd or at least 100 bpd of green crude. In some embodiments, the IBR produces at least 50 bpd or at least 60 bpd of diesel and at least 30 bpd of jet fuel. In some embodiments, the IBR produces at least 80 bpd or at least 90 bpd of diesel. In some embodiments, the IBR can use between approximately 1.0 to 3.0 MW of energy to produce at least approximately 80 bpd of fuel. In some embodiments, the IBR uses less than approximately 2.5 MW or less than approximately 1.75 MW of energy to produce at least approximately 80 bpd of fuel. The IBR can also be self-sustaining, by using a fraction of the fuel it produces to generate enough energy to operate the IBR.

The present disclosure also provides an IBR with a production unit that is an open pond, greater than 20,000 acres, receives a water input greater than 20,000 acre-ft/yr, receives greater than 500,000 SCFD of H₂, or receives approximately 150,000 MT/yr of CO₂; and produces greater than 10,000 bpd green crude. In some embodiments, the IBR can produce greater than 9,000 bpd of a fuel composition. In some embodiments, it produces at least 5,000 bpd of diesel and at least 4,000 bpd of jet fuel. In some embodiments, at least 9,000 bpd of diesel is produced. The CO₂ sequestering, or CO₂ capture of the IBR can be approximately 4,000 MT/day. In some embodiments, the IBR can use between approximately 100 to 200 MW of energy to produce at least approximately 9,000 bpd of a fuel composition. In some embodiments, the IBR uses less than approximately 125 MW or less than approximately 175 MW of energy to produce at least approximately 9,000 bpd of a fuel composition. The IBR can also be self-sustaining, by using a fraction of the fuel it produces to generate enough energy to operate the IBR.

The systems disclosed here also provide methods for earning carbon credits. For example, provided herein is a method for earning carbon credits comprising growing modified or unmodified organism near a refinery and refining oil from the organism in the refinery. In some embodiments, the refinery is within 500, 250, 100, 75, 50, 25, 10 or 5 miles from the production unit for growing the organism. Another method of earning carbon credits provided herein is a method comprising: growing an organism in a production unit, wherein the organism sequesters at least approximately 50 MT/day, or at least approximately 4,000 MT/day of CO₂, extracting an oil composition from the organism; and, obtaining carbon credits from the sequestering. In some embodiments, the carbon dioxide, or a portion of it, is produced by a refinery.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Many novel features of the invention are set forth with particularity in the appended claims. A better understanding of exemplary features and advantages of the invention will be obtained by reference to the following detailed description that sets forth illustrative embodiments, in which many principles of the invention are utilized, and the accompanying drawings of which:

FIG. 1 depicts a schematic of a system for biorefining and fuel production.

FIG. 2 depicts a schematic of a system for biorefining and fuel production using algae.

FIG. 3 depicts a schematic of an exemplary Integrated Biorefinery (IBR) using algae.

FIG. 4 depicts a schematic of an exemplary smaller scale IBR using algae.

FIG. 5 illustrates a hexane extraction process adapted for algae.

FIG. 6 depicts a comparison of the composition of crude oil and algae oil.

FIG. 7 depicts a schematic of refining to generate green diesel, naphtha, propane, and jet fuel.

FIG. 8 depicts the chemical process of hydrotreating triglycerides resulting in N-paraffinic products.

FIG. 9 illustrates oil products made by algae that did not initially have the ability to produce these oil products. SE base is the strain with an introduced gene fusicoccadiene synthase.

FIG. 10 illustrates the algal strain depicted in FIG. 9 can be systematically developed to improve its ability to produce the oil by directed evolution and mutagenesis.

FIG. 11 illustrates an example of an algal gamete produced in the laboratory of an algal species that does not naturally breed.

### DETAILED DESCRIPTION

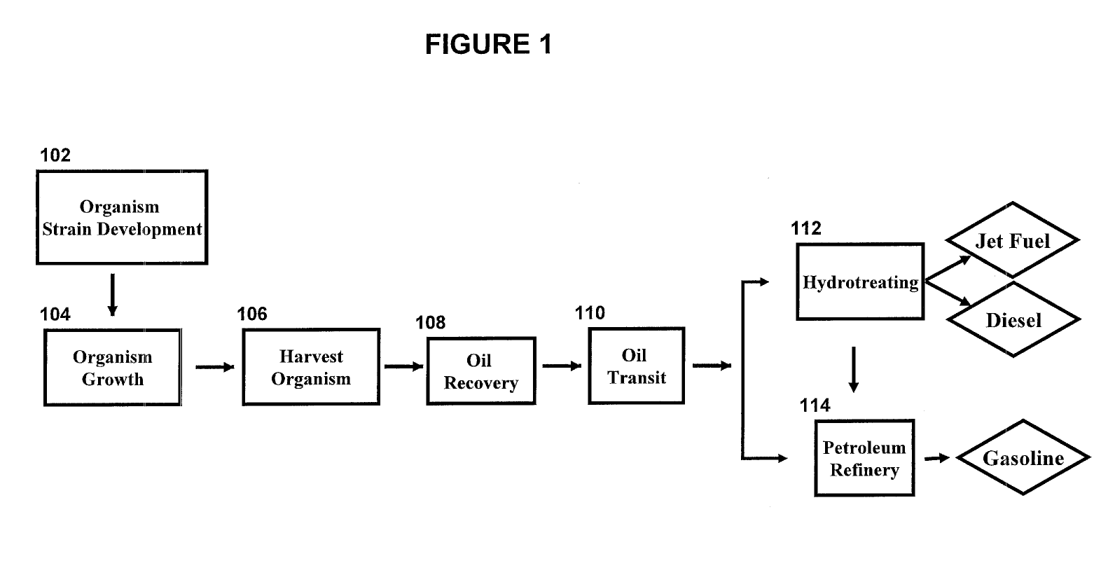

Disclosed herein are systems and methods of generating biofuels. The systems for generating biofuels can be integrated and are referred to herein as Integrated Biorefineries (IBRs). An IBR has various elements with inputs and outputs that are interconnected, such that a resulting product or byproduct from one unit is inputted to another unit through various conduits leading from one unit to another. The units can be adjacent or in close proximity to each other. Alternatively, the units are not adjacent to each other. The various units in the IBR can be operated by a single entity, or different entities. The systems use biofeedstock from an organism grown in the IBR to generate fuel products. An IBR can use a single biofeedstock to generate diesel fuel, jet fuel and gasoline, such as using algae as the biofeedstock. The IBRs can also be used to obtain carbon credits and be self-sustaining. Examples of IBRs are illustrated in FIG. 1-4, with the systems described in more detail below.

The systems herein include those, such as disclosed in FIG. 1. Such system performs the following steps: developing an organism strain (e.g., a microalgae) with improved property(ies) (e.g., high salt tolerance, herbicide resistance, pest resistance, ability to grow in high pH, improved utilization of nitrogen, temperature stability, and characteristics for dewatering, flocculating ability) (102), growing the organism in an open pond or closed bioreactor (104), harvesting the organism (e.g., by flocculating the cells) (106), recovering a product such as an oil composition (e.g., fatty acids, triglycerides, and/or terpenes) from the organism (108), transporting the oil composition (e.g., green crude) to one or more refineries (110) (e.g., via trucks or pipelines), and refining the oil composition to produce one or more fuels (112 and 114), such as jet fuel, diesel fuel, and/or gasoline. Different fuel products can be produced by the system simultaneously or in series. For example, the system can include a hydrotreating plant or unit (112) that can convert the green crude to jet fuel and diesel. The system can also include a petroleum refinery (114) that can convert the crude oil and products from the hydrotreating plant to gasoline. For example, the production of jet fuel and diesel fuel can result in additional products, such as naphtha and light hydrocarbons, such as propane, that are then used for generating gasoline. Exemplary light hydrocarbons include, but are not limited to, methane, ethane, propane, and butane. In another example, production of gasoline can result in additional products, such as diesel, that are used for producing jet fuel.

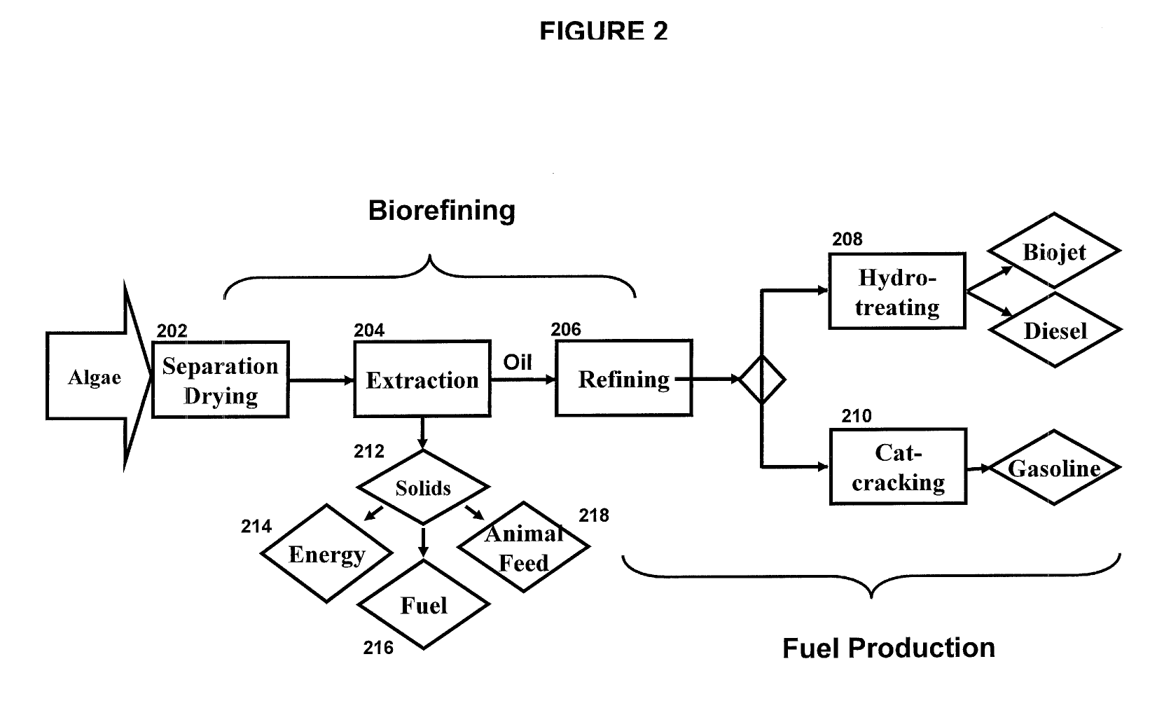

In some embodiments, the systems disclosed herein use algae as the organism (FIG. 2). The algae can be harvested and separated from the culture media, resulting in an algal paste. The algae or algal biomass may optionally be dried (202) prior to performing dry extraction. In some instances the algae remains wet to some extent and need not be fully dewatered before extraction occurs. Algal oils are then extracted from the biomass and are separated from algal solids (204). Extraction may utilize hexane in processes such as those described in more detail herein or other hexane extraction methods known in the art.

The oil composition can then be refined (206). Optionally, refining can involve removal of contaminants. For example heteroatoms and metals can be removed by hydrotreating (e.g. hydrodenitrogenation (HDN), hydrodeoxygenation (HDO), and/or hydrodemetallization (HDM)). Hydrotreating of the oil composition can produce jet fuel and/or diesel (208). The oil composition can also be refined by catalytic cracking (210) to produce gasoline. The refining by hydrotreating and catalytic cracking can occur concurrently (both processes occurring) or alternatively (one or the other is occurring). The refining processes can also be subsequent to each other, for example, products produced by hydrotreating (208), can then be processed by catalytic cracking (210). Products from one refining process (e.g., H₂) can also be further used by another refining process. The refining processes can be separate units of the system, or in the same unit. Moreover, the algal solids (212) can be used to produce fuels (216), such as ethanol by enzymatically breaking cellulose; animal feed (218), by adding one or more components to the animal feed, such as biomass degrading enzymes (e.g. a carbohydrase, protease or lipase) or nutrients (e.g. tocopherols); and/or energy (214), such as methane gas released from digestion of the solids.

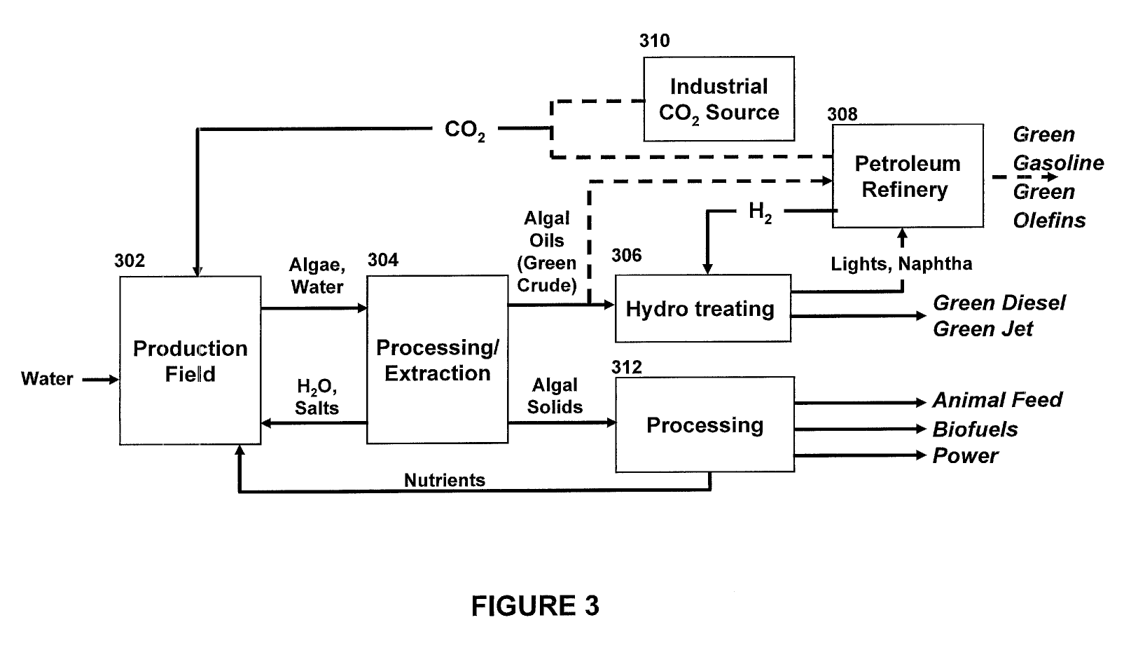

In some instances, the systems herein comprise units that are interconnected, such as depicted in FIG. 3. For example, an IBR can comprise a growth or production unit (302), a processing unit for extraction (304), a first refining unit for generating diesel and jet fuel (306), a processing unit for processing solid extracts from the organism (312), a second refining unit (308), and optionally a CO₂ source (310). Each unit is connected to another unit within the IBR by either receiving an input from another unit, or producing a product that is inputted into another unit, or both receiving an input from another unit and producing a product that is inputted into another unit. The units can be adjacent, or in close proximity, to each other, not adjacent to each other, or some units are adjacent to another unit, while other units are not. For example, adjacent can be within approximately within 5, 10, 15, 20, 30, 40, 50, or 100 miles within each other, for example a refinery can be approximately 500, 250, 100, 75, 50, 25, 10 or 5 miles from the production unit. The various units can be operated by a single entity, or different entities. The IBR can also use a single biofeedstock from an organism grown the growth unit, or more than one biofeedstock may be used by the IBR. The IBR can produce a variety of fuel products concurrently. The IBR can also be used to obtain carbon credits and be self-sustaining, by generating enough power or fuel products to be used to operate the IBR, while also producing additional fuel products that can be sold. The production field or growth unit (302) generally requires water, salts, nutrients, such as phosphorus, nitrogen, sulfur and trace minerals, and CO₂ for growing and maintaining the organism. For example, the nutrients can be in any form usable by algae, for example, ammonia, nitrates, phosphates, and CO₂. When using an open pond system for a production unit, such as raceway style ponds (e.g. Oswald ponds from Pond Treatment Technology, by Andy Shilton), the inputs of water, salts, nutrients, and CO₂ can all be supplied from external sources and/or from other units of the IBR. For example, CO₂ can be supplied from local cement refineries, coal burning plants, or from a CO₂ pipeline. In some instances the CO₂ used is atmospheric CO₂. In some instances, a combination of atmospheric CO₂ and other sources is used. When an IBR is partially or totally integrated, water, salt, nutrients, and/or CO₂ can be provided to the production unit from other units of the IBR. For example, water and salts resulting from the unit for processing and extracting products (304) can be directed to the production unit. In a further example, nutrients from the processing unit that processes the solid extracts from the organism (312) can also be directed back to the production unit. Furthermore, CO₂ from the petroleum refining unit (308) can be directed back to the production unit. Optionally, an additional unit can be provided to supply CO₂. Such additional unit can be a CO₂ pipeline, another refinery, or other industrial CO₂ source. CO₂ can be transported as a gas or in liquid form by pipeline or truck depending on the amount needed.

The output of the production field (302) is the organism which is harvested for processing by the next unit (304). An oil composition and solid extract comprising hydrocarbons, lipids, fatty acids, aldehydes, alcohols, alkanes, or combinations thereof can be extracted from the harvested organism by methods as described herein. Both the oil composition and the solid extracts resulting from the extraction can then be used for subsequent processing within the IBR (306, 312). The processing/extraction can also produce water and salts which can be inputted back into the production unit (302).

The oil composition can be refined by a refinery for hydrotreating (306) to produce diesel and jet fuel, refined by a refinery to produce gasoline or olefins (308), or both. In some embodiments, the oil composition can have heteroatoms removed prior to other refining process, such as cracking or isomerization. Alternatively, the oil composition to be refined more than once, for example, light hydrocarbons, with low molecular weight such as methane, ethane, propane and butane, and naphtha can be produced from hydrotreatment (306) and can be subsequently refined to produce gasoline or olefins (308). The refining units can also produce products that are inputted back into the IBR. For example, the refining unit can generate CO₂ and H₂ (308) which can be inputted into the production field (302) and other refining units (306), respectively. In some instances, ammonia products can be generated by the refining process and recycled as a nutrient for growth of the organism. The IBR can also comprise an additional CO₂ source that also inputs CO₂ into the production field. Furthermore, the IBR can be a system for sequestering CO₂ which can be used to obtain carbon credits, discussed further herein. The solid extracts produced can be processed to generate animal feed, biofuels, and power (312). For animal feed, the solids can be dried and pelleted or fed wet if a animal facility is nearby. The solid extracts can also be digested to produce methane and CO₂ with the methane used for fuel and the CO₂ recycled back to the production unit. Dried biomass can be directly burned for power and the CO₂ recycled. Biomass can also be converted into liquid fuel by hydrous pyrolysis or the production of syngas (CO and H) which is converted to liquid fuels by the Fishcer-Tropsch process. The biomass can also be anaerobically digested and/or aerobically digested and the nutrients, such as phosphorous, nitrogen, sulfur, and potassium can be put back in the production unit to decrease external inputs. Thus, the processing can generate nutrients that are inputted back into the production field (302).

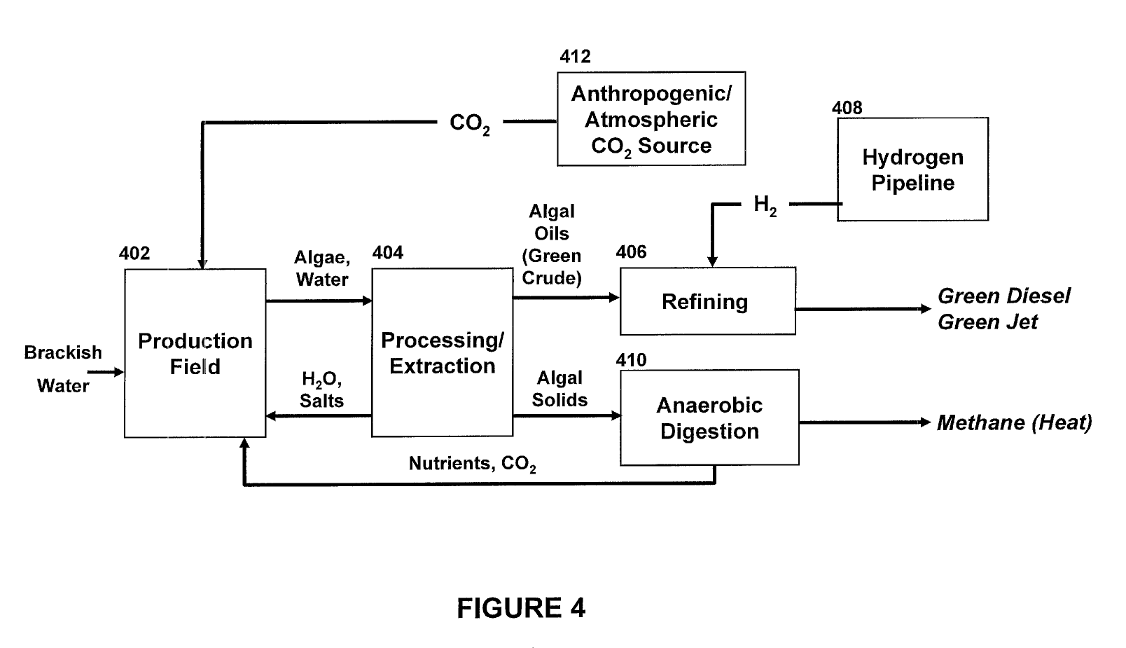

The IBR can also be as depicted in FIG. 4. The IBR can comprise a growth or production unit (402), a processing unit for extraction (404), a refining unit for generating diesel and jet fuel (406), a H₂ source (408), a processing unit for processing solid extracts extracted from the organism (410), and a CO₂ source (412). As described, the production field (402) can obtain water, salts, nutrients and CO₂ for growing and maintaining the organism from other units of the IBR. For example, the water and salts result from the unit for processing and extracting products from the organism products to be used for producing fuels (404). The nutrients and CO₂ can be from the processing unit that processes the solid extracts from the organism (410), the H₂ can be from an external source and supplied by a pipeline (408). For example, the hydrogen can be from refineries, such as resulting from steam/methane reforming of various hydrocarbon compositions. The hydrogen can also be from the biomass. The CO₂ can from the atmosphere or anthropogenic (412). Thus, the IBR can be a system for sequestering atmospheric CO₂, which can be used to obtain carbon credits. As described above, the output of the production field (402) is harvested for processing by the next unit (404). The processing/extraction can also produce water and salts which can be inputted back into the production unit (402).

The oil composition and the solid extracts resulting from the extraction can then be used for subsequent processing within the IBR (406, 410). The oil composition can be refined by a refinery (406) to produce diesel and jet fuel. The solid extracts produced can be processed by anaerobic digestion to methane, which can be used to generate power such as heat (410). Anaerobic digestion using methods known in the arts (e.g. WO 03/042117, US20020079266, US20080311640) can be used to produce methane, that can be burned to heat water or generate electricity. Anaerobic digestion can generate nutrients and CO₂ that are inputted back into the production field (402). The solid extracts can also be processed by fermentation by methods known in the art, (e.g. US20090006280) to produce alcohol, including but not limited to methanol, ethanol, propanol, and butanol, as well as gaseous co-products such as carbon dioxide.

The systems disclosed herein can also be self-sustaining. For example, a fraction of the fuel compositions being produced by the IBR can be used to run the IBR, while the remainder can be sold. For example, at least approximately 5, 10, 15, 20, 25, 30, 35, 40, 45, 50, 55, 60, 65, 70, 75, 80, 85, or 90% of the fuel it produces is used to generate the energy for operating the IBR, and the leftover fuel can be sold to a third party. In some embodiments, the IBR generates at least approximately 5, 10, 15, 20, 25, 30, 35, 40, 45, 50, 55, 60, 65, 70, 75, 80, 85, 90, 100, 110, 120, 130, 140, 150, 200% of its own energy needs. The IBR may generate enough power to run itself by using power not from the oil compositions obtained from the organism, but from the resulting byproducts, such as solid extracts that remain after extraction, which can be used to generate power. Alternatively, the power may generated from both the fuel compositions obtained from the microorganism and the resulting byproducts. The solid extracts can be processed by anaerobic digestion, aerobic digestion or both to produce biofuels such as methane or ethanol, which can then be used to power the IBR.

Further details of the various units of the IBRs are discussed below.

### Organisms

The organisms used in the IBRs disclosed here, such as those developed for use (102, FIG. 1), can be photosynthetic, either naturally or genetically modified to be photosynthetic. The organism can be a microorganism. The organism can be unicellular, non-vascular, or both. For example, the microorganism can be algae, or green algae such as of the genus Chlamydomonas. The microorganism can be a Chlamydomonas sp, a Dunaliella sp, a Haematococcus sp or a Scenedesmus sp, for example, C. reinhardtii, D. salina, H. pluvalis, S. dimorphus, D. viridis, or D. tertiolecta. The algae can also be of the genus Chlorella. In some embodiments, the microorganism is bacteria, such as cyanobacteria or any other bacteria of the genus Synechocystis, Synechococcus, or Athrospira. The microorganism can be a cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, or phytoplankton. In some instances, the organism is any organism or microorganism other than c-chlorophyll containing algae.

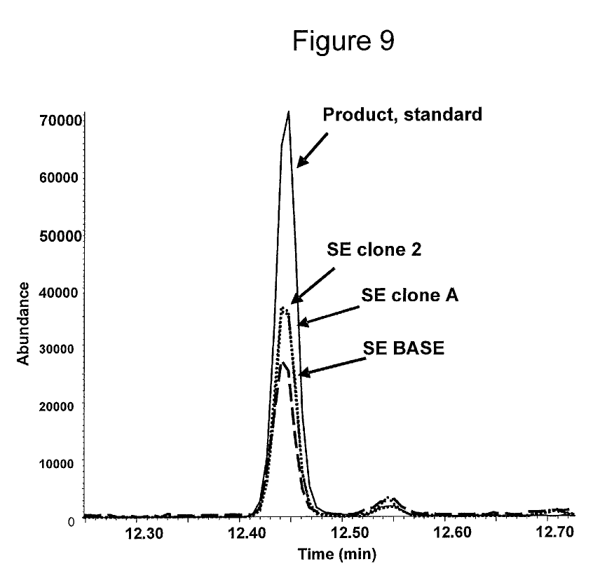

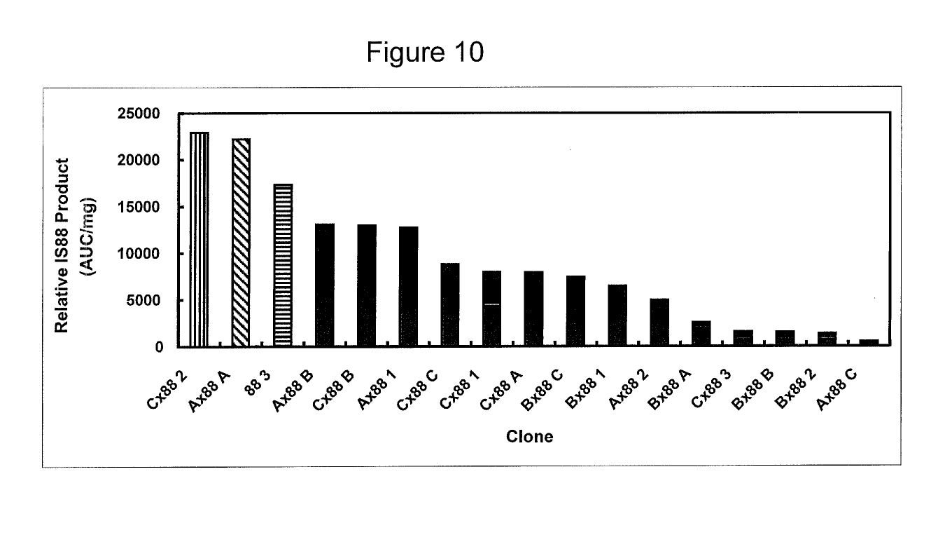

The development of organisms (102, FIG. 1) for use in IBRs includes developing strains that can be cultivated in commercial environments, such as the production units disclosed herein (104, FIG. 1, 302, FIG. 3, 402, FIG. 4). Commercial cultivation places emphasis on growing an organism with the desired trait(s), protecting its growth during its cultivation cycle; using cost-effective, optimized nutrients to improve yield; and cultivating the organism such that efficient, large-scale harvesting can be performed. In some embodiments, development of strains includes evaluating species of the organism. For example, the organisms are collected, screened, and measured for commercial traits (e.g. environmental tolerance, herbicide resistance, salt tolerance, temperature tolerance, pH tolerance, yields of desired products, pest resistance, improved utilization of nitrogen, improved characteristics for dewatering, flocculating ability). The evaluations can be used to provide an informed basis for developing an organism with an improved ability to be commercially cultivated, such as having an improved ability to produce fuels. Furthermore, a selected strain with an improved ability or trait can be systematically improved using directed evolution and mutagenesis techniques. For example, an algal strain with improved ability to produce an oil (FIG. 9) is developed to have increasing ability to produce the oil by directed evolution and mutagenesis techniques (FIG. 10).

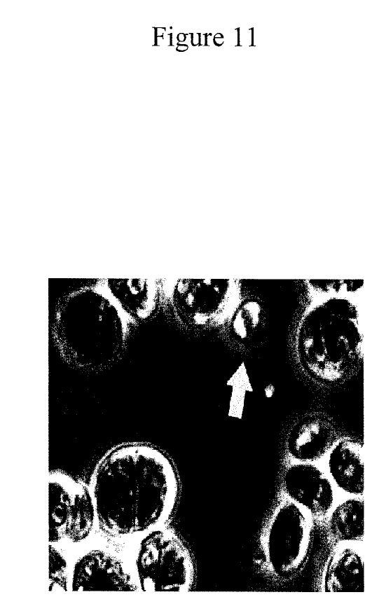

The organism can have an improved ability to produce fuel products (102, FIG. 1). The organism can be naturally occurring and selected for specific or desired property(ies), characteristic(s) or trait(s) that improve its ability to produce fuel products. The organism can also be genetically modified to have the desired property(ies) or characteristic(s). The characteristics selected or genetically modified can include, but not be limited to, increasing the production of a product e.g., hydrocarbons, lipids, fatty acids, aldehydes, alcohols, alkanes, isoprenoids or combinations thereof useful for fuel production. The characteristic selected or genetically modified can also include increasing the tolerance of the organism to grow in selected environments, e.g., higher salt tolerance or herbicide resistance. For example, an algal strain was modified to have increased tolerance to specific commercial environmental conditions. The characteristic selected or genetically modified can also include improving the harvesting or collection of the organism or its product useful for fuel production, e.g., ability to flocculate or dewater. Another characteristic can be the ability for the organism to breed (see for example FIG. 11, an algal species that is reported to be resistant to breeding that was induced to produce a gamete, as indicated by the arrow). Any of these characteristics can be combined in a single strain of an organism. For example, an algal strain may have been developed to have increased salt tolerance, but the strain cannot be bred. The strain can then be developed to breed. One or more traits can be developed concurrently with one or more other traits, or subsequent to the development of one or more other traits. For example, an algal strain can be developed to have herbicide resistance to two different agents. In other embodiments, the characteristic may be an increased ability to secrete a product, such as hydrocarbons, hydrocarbons, lipids, fatty acids, aldehydes, alcohols, alkanes (e.g. terpenes, isoprenoids, triglycerides, etc). For example, an organism may be genetically modified to secrete isoprenoids.

Improving an ability of an organism can include increasing a characteristic the organism already has. For example, a microorganism can tolerate growing in salt conditions; the microorganism can be modified to increase its salt tolerance. Alternatively, improved abilities can include giving a characteristic to the organism that the organism did not originally have. For example, a microorganism did not have the ability to produce a particular hydrocarbon. The microorganism can be modified to produce the particular hydrocarbon. The development of an organism can be for any one characteristic or any combination as described herein. The development of an organism may comprise random mutagenesis and selection of an organism with a particular characteristic, such as improved ability to produce a fuel product. For example, genetically modifying an organism can be by directed evolution, where a gene of interest is mutated or recombined at random to create a large library of variants (e.g. by low fidelity PCR or DNA shuffling). The library is then screened for the presence of the mutants/variants with the desired trait(s) (e.g. increased ability to produce a hydrocarbon or oil, increased environmental conditions tolerance). The identification of the mutants/variants with the desired trait(s) is the amplified and analyzed (e.g. by sequencing). Many rounds of this can be performed. Any of the techniques may be used concurrently, or subsequent to other techniques. For example, an organism may first be genetically modified. Alternatively, the organism may be genetically modified with a known genetic modification to have an increased ability to produce a fuel product as compared to an unmodified organism. The techniques can be combined in developing an organism for use in an IBR. For example, an organism may be modified by transformation with an expression vector and then undergo directed evolution. Alternatively, an organism may undergo directed evolution prior to transformation with an expression vector.

An organism can be modified through the use of expression vectors. For example, the organism can be modified by nuclear transformation. For organisms with chloroplasts, such as algae, chloroplast transformation may be performed. In some embodiments, the organism can have its entire chloroplast genome or entire genome replaced. The organism may be genetically modified such that expression of a gene or product is regulated, such as inducible. One or more or all cordons of an encoding polynucleotide can also be biased to reflect a particular organism's preferred codon usage. For example, if the organism is algae, the expression vector typically comprises a gene that is to be expressed in the algae with a codon bias favored by the algae, such as the codon bias of C. reinhardtii as described in U.S. Publication Application 2004/0014174.

The organism can have increased salt tolerance, herbicide resistance, increased pH tolerance or combinations thereof. For example, the organism can be genetically modified to have increased salt tolerance, such as being able to grow in a high-saline environment of at least approximately 0.5, 1.0, 1.5, 2.0, 2.5, 3.0, 3.5, or 4.0 molar sodium chloride. The organism may have increased salt tolerance being transformed with an expression vector that encodes a transporter or a protein that regulates the expression of a transporter. The transporter can be an ion transporter, such as an ATPase, including, but not limited to Na+ ATPase or a P-type ATPase. The ion transporter can also be an antiporter, such as a Na+ antiporter. Examples of the antiporter include but are not limited to NHX1 or a component of the SOS pathway. A component of the SOS pathway can be SOS1, SOS2, SOS3, or a functional homolog thereof. The organism can also be genetically modified to have increased salt tolerance by being genetically modified by an expression vector for an H+-pyrophosphatase, such as AVP1 or a functional homolog thereof. In some embodiments, the polynucleotide encodes a protein that regulates the expression of a transporter.

The organism may be genetically modified to express or increase the expression of one or more herbicide or insect resistance-conferring proteins. For example, the organism can be transformed with a polynucleotide that encodes a protein that is toxic to one or more animal species, such as a gene encoding a Bacillus thuringiensis (Bt) toxin that is lethal to insects. A glyphosate resistant organism can be developed by being genetically modified to express mutant 5-enolpyruvylshikimate-3-phosphate synthase (EPSPS). In another instance, the organism can express glyphosate oxidoreductase (GOX), a glyphosate acetyl transferase (GAT), or an EPSP synthase.

The organism can have an increased ability flocculate, providing increased ability to collect and harvest the organism for use in producing a fuel composition. For example, the organism can also be genetically modified to produce a flocculating moiety such as a carbohydrate binding protein, antibody, lectin, FhuA protein, or pb5 protein. Expression of the flocculation moiety is preferably inducible.

The organism can produce or increase production of a hydrocarbon, steroid, fatty acid, lipid, oil, or any combination thereof. For example, an organism can be modified to have an enriched profile for a specific type of hydrocarbon as compared to the original strain. For example, the organism may have an improved ability to produce a terpene, isoprene, or isoprenoid. The organism may have an improved ability to produce an isoprenoid with two phosphates, such as GPP, IPP, FPP, GGPP or DMAPP. The organism can also be developed to produce or increase production of a lipid, such as triglycerides. For example, an organism can be modified to produce oils that it was unable to produce prior to modification, such as shown in FIG. 9. For example, the organism may be genetically modified to express a lipase. The organism may express acetyl-CoA carboxylase, ketoreductase, thioesterase, malonyltransferase, dehydratase, acyl-CoA ligase, ketoacylsynthase, enoylreductase or a desaturase. To increase the ability of the organism to produce a hydrocarbon, steroid, fatty acid, lipid, the lipid synthesis pathway or isoprenoid production pathway may be modified in the organism.

The organisms can also have an improved ability to degrade a biomass. For example, the organism can have an increased ability to produce a biomass degrading enzyme such as, but not limited to, an exo-β-glucanase, endo-β-glucanase, β-glucosidase, endoxylanase, or ligninase.

The organisms described here can have a limited number of life cycles, such as an algal cell with a limited number of life cycles. The number of life cycles can be the natural number of life cycles of the organism, or the number of life cycles can be from selecting and developing an organism with that number of life cycles. The number of life cycles can be between approximately 5-100 life cycles. In some embodiments, the number of life cycles can be between approximately 5-100, 10-150, 5-25, or 5-10 life cycles.

Different organisms, species, or strains of organisms may be used in the production area of the IBR disclosed herein at different times of the year. In some embodiments, the same organism, species, or strain is used year round. For example, a specific algal strain s may be used in certain times of the year and another strain for other times of the year. On strain may be used in the warmer season, such as summer, versus another strain, used for the winter.

### Production Unit

The developed organism is then grown and maintained in a growth area or production field (104). The growth area provides an environment conducive for growing, culturing, or maintaining a population of the selected organism, such as algae. Maintaining a population can include periodic supplementing of the growth area with seeder cultures of the selected organism. For example, a growth area is conducive for maintaining a population of algae, however the algae has a limited number of life cycles, as a result the growth area is supplemented with starter or seeder cultures of the algae that can grow in the growth area. The growth area may be adjacent or in close proximity to a number of smaller growth areas (e.g. within 5, 10, 15, 20, −30, 40, 50, or 100 miles within each other, for example, less than approximately 5, 10, or 15 miles). The smaller growth areas can be used to grow started culture, or seeder cultures, of the organism, which is then used to inoculated or seed the larger growth area or production unit.

The growth area can be exposed to natural light, such as sunlight, or to artificial light. The growth areas can receive simultaneous and/or alternating combinations of natural light and artificial light. The growth area can open or closed. Open growth areas can be naturally exposed to sunlight, exposed to artificial light (for example if the open area is an area that receives little or no sunlight), or to both natural and artificial light (such as receiving light during the day, and artificial light at night). The number of photons striking the organisms can be manipulated, as well as other parameters such as the wavelength spectrum and ratio of dark:light hours per day. The growth area can also have its salinity, pH, temperature, or various other parameters controlled or manipulated.

The growth area, such as an open growth area, can comprise at least approximately 1, 5, 10, 100, 200, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 22,000, 30,000, 40,000, or 50,000 acres. The pH of the production area, such as an open pond, can be between approximately 5-12, 6-11, 7-11, 8-11, 9-11, 10-11, 8-10, or 8-9. The salinity of the production area, such as an open pond, can be brackish to hypersaline. For example, the salinity can contain between approximately 0.5 to 30 grams of salt per liter (about 0.5 to 30 parts per thousand, or ppt). In other embodiments, the salinity may be between approximately 30-50 ppt. In some embodiments, the salinity is between approximately 20-50 ppt, 20-40 ppt, 30-40 ppt, 25-40 ppt, or 30-35 ppt.

Open growth areas or production areas can be non-arable land. Open growth areas can be open ponds, lakes, or any other body water. Open growth areas can be natural, such as a pre-existing pond, or artificial, such as constructed by humans. For example, an open pond for growing and maintaining algae can designed and constructed on non-arable land. The open growth area can be in an area that is dry or desert-like, have high salinity, or have extreme pH. In some instances, light introducers can be added to the ponds to direct light deeper into the ponds.

In some embodiments, the growth area or production unit is a raceway pond. For example, the raceway pond can have the organism, water and nutrients circulate around a racetrack. Paddlewheels can provide the flow and keep the organism suspended in water, and allow the organism to be circulated back up to the surface on a regular frequency, particularly for organisms that are photosynthetic. For photosynthetic organisms, such as algae, the growth area can be kept shallow to allow the organism to be exposed to sunlight. The size of the ponds can be measured in terms of surface area (as opposed to volume), since surface area is generally critical to capturing sunlight. The productivity of the organism can then be measured in terms of biomass produced per day per unit of available surface area. The ponds can operated continuously; that is, water and nutrients are constantly fed to the pond, while organism-containing water can be constantly removed at the other end. In some embodiments, the pond has a semi-permeable barrier on the bottom of the ponds. The temperature of the production area that is open, such as an open pond system, is that of its surrounding environment. For example, an outside pond can have an ambient temperature.

The IBR can comprise more than one growth area or production unit. For example, the IBR can comprise a first growth area and a second growth area, such that when or if the first growth area requires maintenance or cleaning, the second growth area can be used. The first and second growth areas may be connected such that when the first growth area cannot be used, any organisms in the first growth area can be transferred to the second growth area. The IBR can comprise more than two growth areas. In some embodiments, the additional growth areas may be used for growing one or more seeder cultures (such as described herein) or as one or more “back up” growth areas. For example, an IBR can comprise two open ponds, one being used a production area for algal and the second pond does not comprise any organism. When the first production pond needs to be cleaned, or undergoes routine maintenance, the algal can transferred from the first open pond to the second open pond. The IBR can comprise additional open ponds, such as for a seeder culture of algae, or additional production units.

The growth area can also be closed, such as a complete enclosure or partial enclosure. The growth area can be a pond system on outdoor land, but enclosed or partially enclosed. Alternatively, the growth area can be completely enclosed in a bioreactor, such as described in US20050260553. The bioreactor can receive artificial light, natural light, or both, simultaneously or alternating artificial and natural light. For example, the growth area can be exposed to one or more light sources to provide an organism, such as algae, with light as an energy source via light directed to a surface of the bioreactor. Preferably the light source provides an intensity that is sufficient for the organism to grow, but not so intense as to cause oxidative damage or cause a photoinhibitive response. In some instances a light source has a wavelength range that mimics or approximately mimics the range of the sun. In other instances a different wavelength range is used.

To maintain the production field or growth area, such as gases, solids, semisolids, liquids, or combinations thereof, are needed to sustain an environment for maintaining and growing the organism. The inputs can be from an external source or from within the system as part of the IBR. Inputs, such as CO₂, water, salts, and other nutrients, can be generated from within the IBR, such as from processing units within the IBR, and used by the production field. For example, CO₂, water, salts, and other nutrients can be generated as byproducts from the recovery or extraction of products (e.g. oil composition) from the organism. The subsequent processing of the extracted products, such as anaerobic digestion, aerobic digestion or both can also generate nutrients for the production field.

The source of CO₂ for the production field can be an atmospheric source, industrial source, anthropogenic source, or combinations thereof. For example, CO₂ may be from flue gas, and in particular flue gas produced from the combustion of fossil fuels. The CO₂ can be supplied by a gas-to-liquids plant, such as a refinery, a waste water treatment plant, slaughter house, food production facility, grain processing facility, ethanol plant, pulp plant, or paper plant. The CO₂ source can be a unit of the IBR, such as in close proximity to the production unit (e.g. within approximately 5, 10, 15, 20, 30, 40, 50, or 100 miles). For example, the CO₂ source can be a waste water treatment plant that is adjacent to the growth area. The CO₂ generated is released in the nearby atmosphere of the growth area and used by the organisms in the adjacent growth area. Alternatively, CO₂ generated from the waste water treatment plant can be directed into the growth area with a pipe or similar means connecting the plant and the growth area. In another embodiment, the CO₂ source is a refinery that is used to refine the products produced by the organism in the growth area. The CO₂ generated is released in the nearby atmosphere of the growth area or directed into the growth area with a pipe connecting the refinery and the growth area.

Entry and exit of gas, solid, semisolid and liquid input into and out of the growth area containing the organism can be through a port. Ports refer to an opening in the growth area that allows influx or efflux of materials such as gases, liquids, and cells and are connected to tubing, pipelines or other means of conveying substances from the growth area. The port of a growth area can also be used for sampling the culture. A sampling port can be configured with a valve or other device that allows the flow of sample to be stopped and started. Alternatively a sampling port can allow continuous sampling. The growth area can also have at least one port that allows inoculation of a culture. Such a port can also be used for other purposes such as media or gas entry. The use of ports typically allow for greater manipulation of the amount and type of input into the growth area.

Gas can also be introduced by diffusers, e.g. bubblers, or added to water before it goes into ponds by introduction into pipes carrying water to ponds or by treating the water with the gas, such as CO₂ in tanks before putting into the pond or some combination of the preceding. For example, the use of at least some of the CO₂, such as CO₂ generated from a petroleum refinery, involves dissolving CO₂ in an aqueous solution that is then inputted into the production unit. In some such embodiments, the aqueous solution comprises caustic (pH>7) and/or saline water. In other embodiments, the use of at least some of the CO₂, such as CO₂ generated from a plant, involves sequestering the CO₂ through the use of gas separation membranes (e.g. US20020020666) and subsequently delivering the sequestered CO₂ to the production unit.

Closed growth areas have one or more ports allowing entry of inputs and exit of outputs. Open growth areas, or partially enclosed growth areas, can also have ports or may have no ports. For example, an open growth area, such as an open pond, may not have any ports. The inputs can be directly received through the surface of the pond and the outputs directly collected from the pond surface. A combination of one or more ports and open access can also be used open growth areas and partially enclosed growth areas. For example, an open growth area can have ports only for inputs. An open growth area may have a port for inputting gases, nutrients, and water, but no port for collecting any of the microorganisms or its products as they are collected through the surface of the pond. Alternatively, open growth areas and partially enclosed growth areas may have ports only for outputs. For example, the open pond growth area may not have any ports for inputting substances, as they are directly taken in by the pond through its surface, but the open pond system may have a port for collecting the microorganism or its product.

A combination of one or more ports and open access can also be used for the same input in an open growth areas and partially enclosed growth areas. For example, an IBR comprises an open pond growth area and a CO₂ generating refinery. The input of CO₂ is both through atmospheric CO₂ and CO₂ from the refinery that is inputted into the growth area through a port. A combination of one or more ports and open access can also be used for the different inputs in an open growth area and partially enclosed growth area. For example, an IBR comprises an open pond growth area and a processing unit for processing and extracting products from the organism. Water and salts generated from the processing module is inputted into the growth area through a port, whereas CO₂ is obtained through atmospheric CO₂.

A combination of one or more ports and open access can also be used for the same output in an open growth areas and partially enclosed growth areas. For example, the output can be the organism itself, such as algae. The growth area is an open pond and the algae can be harvested directly from the pond as well as collected through a port. A combination of one or more ports and open access can also be used for the different outputs in an open growth areas and partially enclosed growth areas.

Various ports can be used for various inputs and outputs, and control the rate, amount or type of input or output. Gas ports, for example, can be used to convey gases into the growth area. For example, gas inlets can be used to pump gases into the bioreactor or an open pond system. Any gas can be pumped in, including air, air/CO₂ mixtures, noble gases such as argon and others. Air/CO₂ mixtures can be modulated to generate optimal amounts of CO₂ for maximal growth by a particular microorganism. Organisms, such as algae, can grow significantly faster in the light under, for example, 3% CO₂/97% air than in 100% air. 3% CO₂/97% air is approximately 100-fold more CO₂ than found in air. For example, air:CO₂ mixtures of about 99.75% air:0.25% CO₂, about 99.5% air:0.5% CO₂, about 99.0% air:1.00% CO₂, about 98.0% air:2.0% CO₂, about 97.0% air:3.0% CO₂, about 96.0% air:4.0% CO₂, and about 95.00% air:5.0% CO₂ can be infused into a growth area.

The rate of entry of gas into a growth area, or the amount of CO₂ captured by the organisms in the growth area can also be manipulated. For example, the production area can have CO₂ sensors that regulate the amount of CO₂ added. For example, the rate of entry of CO₂ can be controlled through a port. Controlling the amount of CO₂ can also be by modulating the CO₂ emission of a CO₂ source, such as a refinery, that is part of the IBR. The amount of CO₂ released can thus be controlled and amount entering the port into the growth area controlled. Alternatively, because of the controlled CO₂ emission, even emission into the atmosphere, may be controlled perhaps more crudely, and because of its proximity to the growth area (e.g. within approximately 5, 10, 15, 20, 30, 40, 50, or 100 miles), the amount captured by the growth area can also be manipulated. The amount of CO₂ inputted can be at least approximately 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000 metric tonnes per year (MT/yr). The utilization of the CO₂ can be at least approximately 30, 40, 50, 60, 70, 80, 90, 95, 96, 97, 98, 99%. High utilization of CO₂ can be by careful control of pH and other physical conditions. For example, higher pH and lower alkalinity can improve the utilization of CO₂. In some embodiments, higher utilization rates can be achieved by using organisms that were developed to have an increased ability of CO₂ utilization.

Pumping gases into a growth area can serve to both feed cells CO₂ and other gases and to aerate the culture and therefore generate turbidity. Increasing gas flow increases the turbidity of a culture of organisms, such as algae. Placement of ports conveying gases into a bioreactor can also affect the turbidity of a culture at a given gas flow rate. The amount of turbidity of a culture varies as the number and position of gas ports is altered. Turbulence can be achieved by placing a gas entry port below the level of the aqueous culture media so that gas entering the growth area bubbles to the surface of the culture. In a closed system, one or more gas exit ports allow gas to escape, thereby preventing pressure buildup. A gas exit port can lead to a “one-way” valve that prevents other external materials, such as contaminating microorganisms, from entering the closed system. The organisms can also be subjected to mixing using devices such as spinning blades and impellers, rocking of a culture, stir bars, infusion of pressurized gas, hydraulic pumps, and other instruments. Water movement can be by pumping, physical agitation (paddles etc.) gravity, tidal flow.

In some instances, cells are cultured in a growth area for a period of time during which the organism reproduce and increase in number, however a turbulent flow regime with turbulent eddies predominantly throughout the culture media caused by gas entry is not maintained for all of the period of time. In other instances a turbulent flow regime with turbulent eddies predominantly throughout the culture media caused by gas entry can be maintained for all of the period of time during which the organism reproduce and increase in number. In some instances a predetermined range of ratios between the scale of the growth area and the scale of eddies is not maintained for the period of time during which the organisms reproduce and increase in number. In other instances such a range can be maintained.

The growth area can also have one or more ports that allow media entry. Alternatively, media can be inputted not through a port, such as in an open growth area. It is not necessary that only one substance enter or leave a port. For example, a port can be used to flow culture media into the growth area and then later can be used for sampling, gas entry, gas exit, or other purposes. In some instances, such as closed growth areas, the growth area is filled with culture media at the beginning of a culture and no more growth media is infused after the culture is inoculated. In other words, the microorganism is cultured in an aqueous medium for a period of time during which the microorganism reproduce and increase in number; however quantities of aqueous culture medium are not flowed through the growth area throughout the time period. Thus in some embodiments, aqueous culture medium is not flowed through the growth area after inoculation.

In other instances culture media can be flowed though the growth area throughout the time period during which the microorganism reproduce and increase in number. In some embodiments media is infused into the growth area after inoculation but before the cells reach a desired density. In other words, a turbulent flow regime of gas entry and media entry is not maintained for reproduction of microorganism until a desired increase in number of said microorganism has been achieved.

The growth area can also have water inputted. The water can be freshwater, saltwater, or preferably brackish water. The brackish water can be inputted via a port or not through a port. The water inputted can be inputted a rate of at least approximately 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, or 50,000 acre-feet per year (acre-ft/yr). The salinity of the water may be approximately 0.5 to 50. In some embodiments, the salinity may be between approximately 30-50 ppt. In other embodiments, the salinity is between approximately 20-50 ppt, 20-40 ppt, 30-40 ppt, 25-40 ppt, or 30-35 ppt.

In some embodiments, the production unit uses between approximately 0.30 to 100 MW. For example, the production unit may use between approximately 0.30-50 or 0.40-30 MW. The production unit may use less than approximately 100, 75, 50, 40, or 30 MW. In some embodiments, the production unit uses less than 0.50 MW. The production unit may use between approximately 0.20-20 MW for inputting nutrients, water, and gas, into the production unit and approximately 0.1-20 MW for maintaining the production unit (for example, mixing the water in the production unit). In some embodiments, the inputting uses less than approximately 20, 18, 10, 5, 1, 0.30, or 0.26 MW. In some embodiments, maintaining the production unit (for example, mixing) uses less than approximately 15, 12, 11, 5, 1, 0.20, or 0.15 MW.

### Processing Unit

The organism is then collected or harvested from the growth area (106, FIG. 1). The organism may be collected at any density, or when the density of the organism in the pond has reached a specific density. For example, for a sigmoid growth curve, harvesting of the organism may be after the point of curve inflection, but before curve flattens out. The density of the organism may be determined by turbidity or fluorescence of the organism. Harvesting or collection of the organism can also be performed when a product of the organism has accumulated to a particular level.

The organism may be induced to flocculate prior to harvesting. For example, flocculating can be by auto flocculation, such as induction by high pH through the presence of phosphate and divalent cations, or induced by nitrogen limitation and can occur prior to harvesting or collecting the algae. The organism can also be genetically modified to produce a flocculating moiety such as a carbohydrate binding protein, antibody, lectin, FhuA protein, or pb5 protein. Induction of production of the flocculating moiety can occur prior to harvesting.

The organisms can also be harvested and separated from the culture media (such as fermentation broth in a closed system, or pond water in an open system) by centrifugation, and a paste of the organism, or biomass, can be produced. Centrifugation generally does not remove significant amounts of intracellular water from the organisms and thus is typically not a drying step. The biomass, a “wet extract,” can then be washed with a washing solution (e.g., DI water) to get rid of the culture media and debris. Optionally, the washed biomass may also be dried (for example, oven dried, lyophilized, or other means) to produce a “dry extract.” Alternatively, cells can be harvested but not be separated prior to the next step of recovering an oil composition from the cells (108). For example, the cells can be at a ratio of less than 1:1 v/v cells to extra cellular liquid when the cells are lysed and the oil composition extracted. Thus, the algae can be harvested to generate a dry extract or wet extract (such as described in Examples 4 and 5). Harvesting can use between approximately 0.50-150 MW. In some embodiments, harvesting uses less than approximately 150, 110, 75, 50, 46, 40, 30, 20, 15, 2.0, 1.6, 1.5, 1.4, 0.70, or 0.67 MW.

After harvesting, an oil composition, also referred herein as “green crude,” is recovered or extracted from the organism. The green crude can comprise hydrocarbons, lipids, fatty acids, aldehydes, alcohols, and alkanes. Extraction can include dewatering, grinding, crushing, soliciting, homogenizing, solvent extracting, or combinations thereof. Extraction can also include processes for separating various compositions and products produced by the organism, such as separating oil compositions and solid compositions or extracts, obtained from the organism.

The harvested organism is disrupted or lysed to produce a lysate from which the oil composition is recovered. Disruption can be by mechanical means, chemical means, or any convenient means, including, but not limited to, heat-induced lyses, adding a base, adding an acid, using enzymes such as proteases and polysaccharide degradation enzymes such as amylases, using ultrasound, mechanical lyses, using osmotic shock, infection with a lyric virus, or expression of one or more lyric genes. Each of these methods for lysing an organism can be used as a single method or in combination simultaneously or sequentially, to release intracellular molecules which have been produced by the organism. The extent of cell disruption can be observed by microscopic analysis. For example, cell lyses can be at least approximately 70%, 75%, 80%, 85%, 90%, 95%, 96%, 97%, 98% or 99% cell breakage. In some embodiments, the organism is lysed after growth, for example to increase the exposure of cellular lipid, hydrocarbon, or other products for extraction or further processing. For example, the timing of lipase expression, terpene synthase expression (e.g., via an inducible promoter) or cell lyses can be adjusted to optimize the yield of the product, such as lipids, hydrocarbons, or both, to be extracted. A number of lysis techniques, such as described herein a can be used individually or in combination.

In some embodiments, heat-induced lysis is used. For example, a suspension of organisms is heated until the cell walls, cell membranes, or both, of the organisms degrade or breakdown. Typically, temperatures applied are at least approximately 30° C., 50° C., 60° C., 70° C., 80° C., 90° C., 100° C., 110° C., 120° C., 130° C., 140° C. or higher. Lysing cells by heat treatment can be performed by boiling the organism. Alternatively, heat treatment (without boiling) can be performed in an autoclave or other pressurized vessel. The heat treated lysate may be cooled for further treatment. Cell disruption can also be performed by steam treatment, i.e., through addition of pressurized steam. Steam treatment for cell disruption is described, for example, in U.S. Pat. No. 6,750,048.

The organisms can also be lysed using a base. A base can be added to a cellular suspension containing the organism. The base should be strong enough to hydrolyze at least a portion of the proteinacious compounds of the organisms. Bases which are useful for solubilizing proteins are known in the art of chemistry. Exemplary bases which are useful in the methods of the present disclosure include, but are not limited to, hydroxides, carbonates and bicarbonates of lithium, sodium, potassium, calcium, and mixtures thereof. An exemplary base is KOH. Base treatment for cell disruption is described, for example, in U.S. Pat. No. 6,750,048.

The organisms can also be lysed using an acid. An acid can be added to a cellular suspension containing the organism. For example, acid lysis can be effected using an acid at a concentration of approximately 10-500 nM or alternatively 40-160 nM. Acid lysis is often performed at above room temperature e.g., at 40-160° C. or 50-130° C. For moderate temperatures (e.g., room temperature to approximately 100° C. and particularly room temperature to approximately 65° C., acid treatment can usefully be combined with sonication or other cell disruption methods.

The organisms can also be lysed using an enzyme. In some embodiments, the organism may be genetically modified to have inducible expression of an enzyme for lysis. Enzymes for lysing an organism are proteases and polysaccharide-degrading enzymes such as cellulases. For example, enzymes can be polysaccharide-degrading enzymes from Chlorella or a Chlorella virus. Other enzymes that may be used include, but are not limited to, hemicellulase (e.g., hemicellulase from Aspergillus niger; Sigma Aldrich, St. Louis, Mo.; #H2125), pectinase (e.g., pectinase from Rhizopus sp.; Sigma Aldrich, St. Louis, Mo.; #P2401), Mannaway 4.0 L (Novozymes), cellulase (e.g., cellulose from Trichoderma viride; Sigma Aldrich, St. Louis, Mo.; #C9422), and driselase (e.g., driselase from Basidiomycetes sp.; Sigma Aldrich, St. Louis, Mo.; #D9515). Examples of other enzymes can be used for lysis include proteases, such as, but not limited to, Streptomyces griseus protease, chymotrypsin, proteinase K, proteases listed in Degradation of Polylactide by Commercial Proteases, Oda Y et al., Journal of Polymers and the Environment, Volume 8, Number 1, January 2000, pp. 29-32(4), Alcalase 2.4 FG (Novozymes) and Flavourzyme 100 L (Novozymes). Any combination of enzymes can be used concurrently or sequentially.

The organisms can also be lysed by using ultrasound, such as by sonication. Thus, cells can also be lysed with high frequency sound. The sound can be produced electronically and transported through a metallic tip to an appropriately concentrated cellular suspension. This sonication (or ultrasonication) disrupts cellular integrity based on the creation of cavities in cell suspension.

Lysing can also be performed by applying an osmotic shock, or cytolysis, or by mechanical lysis. Cells can be lysed mechanically and optionally homogenized to facilitate collection of a product such as hydrocarbon or lipid collection. For example, a pressure disrupter can be used to pump a cell containing slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. Alternatively, a ball mill can be used. In a ball mill, cells are agitated in suspension with small abrasive particles, such as beads. Cells break because of shear forces, grinding between beads, and collisions with beads. The beads disrupt the cells to release cellular contents. Cells can also be disrupted by shear forces, such as with the use of blending (such as with a high speed or Waring blender as examples), the french press, or even centrifugation in case of weak cell walls, to disrupt cells.

The organisms can also be lysed by infection with a lytic virus. A wide variety of viruses are known to lyse microorganisms suitable for use in the present invention. For example, Paramecium bursaria chlorella virus (PBCV-1) is the prototype of a group (family Phycodnaviridae, genus Chlorovirus) of large, icosahedral, plaque-forming, double-stranded DNA viruses that replicate in, and lyse, certain unicellular, eukaryotic chlorella-like green algae. Accordingly, algae can be lysed by infecting the culture with a suitable Chlorella virus. Methods of infecting species of Chlorella with a Chlorella virus are known. See for example Adv. Virus Res. 2006; 66:293-336; Virology, 1999 Apr. 25; 257(1):15-23; Virology, 2004 Jan. 5; 318(1):214-23; Nucleic Acids Symp. Ser. 2000; (44):161-2; J. Virol. 2006 March; 80(5):2437-44; and Annu. Rev. Microbiol. 1999; 53:447-94.

The organisms can also be lysed by autolysis, where the microorganism is genetically engineered to produce a lytic protein that will lyse the organism. This lytic gene can be expressed using an inducible promoter so that the cells can first be grown to a desirable density, followed by induction of the promoter to express the lytic gene to lyse the cells. The lytic gene can be a gene from a lytic virus. The gene can encode a polysaccharide-degrading enzyme, protease, or other enzymes, such as those described herein. In certain other embodiments, the lytic gene is a gene from a lytic virus. Thus, for example, a lytic gene from a Chlorella virus can be expressed in an algal cell of the genus Chlorella, such as C. protothecoides.

After lysis, products for generating a fuel composition can be isolated from the organism. For example, compositions comprising hydrocarbons, lipids, fatty acids, aldehydes, alcohols, alkanes, or combinations thereof, can be isolated by extraction from the organism. For example, hydrocarbons can be isolated by whole cell extraction. After the cells are disrupted the intracellular and cell membrane/cell wall-associated hydrocarbons as well as extracellular hydrocarbons can be collected from the whole cell mass, such as by use of centrifugation as described above. Various methods are available for separating hydrocarbons and lipids from cellular lysates produced by the above methods.

Lipids and hydrocarbons produced by the organisms can then be recovered by extraction with, for example, an organic solvent. For example, hydrocarbons can be extracted with a hydrophobic solvent such as hexane (see Frenz et al. 1989, Enzyme Microb. Technol., 11:717). Hydrocarbons can also be extracted using liquefaction (see for example Sawayama et al. 1999, Biomass and Bioenergy 17:33-39 and Inoue et al. 1993, Biomass Bioenergy 6(4):269-274); oil liquefaction (see for example Minowa et al. 1995, Fuel 74(12):1735-1738); and supercritical CO₂ extraction (see for example Mendes et al. 2003, Inorganica Chimica Acta 356:328-334).

In some cases, the organic solvent hexane is used for extraction. Typically, the organic solvent is added directly to the lysate without prior separation of the lysate components. In one embodiment, the lysate produced by one or more of the methods described above is contacted with an organic solvent for a period of time sufficient to allow the lipid and/or hydrocarbon components to form a solution with the organic solvent. In some cases, the solution can then be further refined to recover specific desired lipid or hydrocarbon components. In one embodiment, an oil composition is recovered from a biomass that has some moisture, or “wet extract,” such as described in Example 4. Following harvest, the organism can be dewatered and fed to an extraction process to extract oils from the organism. The oil can then be converted to various hydrocarbon fuels utilizing a refining process, further described herein.

The oil composition can be extracted from a biomass, or wet extract, by mixing with hexane and a conditioning agent in a high-shear reactor (see Example 4). The oil is separated from the biomass using high-shear contact with hexane and a conditioning agent. Oil will dissolve into hexane, or other similar solvents, forming a solution called miscella. Water and cellular solids do not dissolve, and can be collected separately from the miscella. Following high-shear mixing, the algae/hexane/water mixture is sent to a decanter where it separates into two distinct liquids: a lighter hexane and oil phase (miscella), and a heavier water and spent solids phase. Miscella from the decanter is fed to a distillation process where algae oil is separated from the solvent. This allows recovery and reuse of the solvent, and purifies the oil to a point where it is ready for downstream processing. Distillation takes advantage of the difference in boiling points of the solvent and oil to separate the two components. The solids in the water phase can be concentrated using a centrifuge or other mechanical concentration equipment. The water removed from the solids can recycled back to the production unit, while the solids, with some residual water, are fed to the solids processing units as described herein.

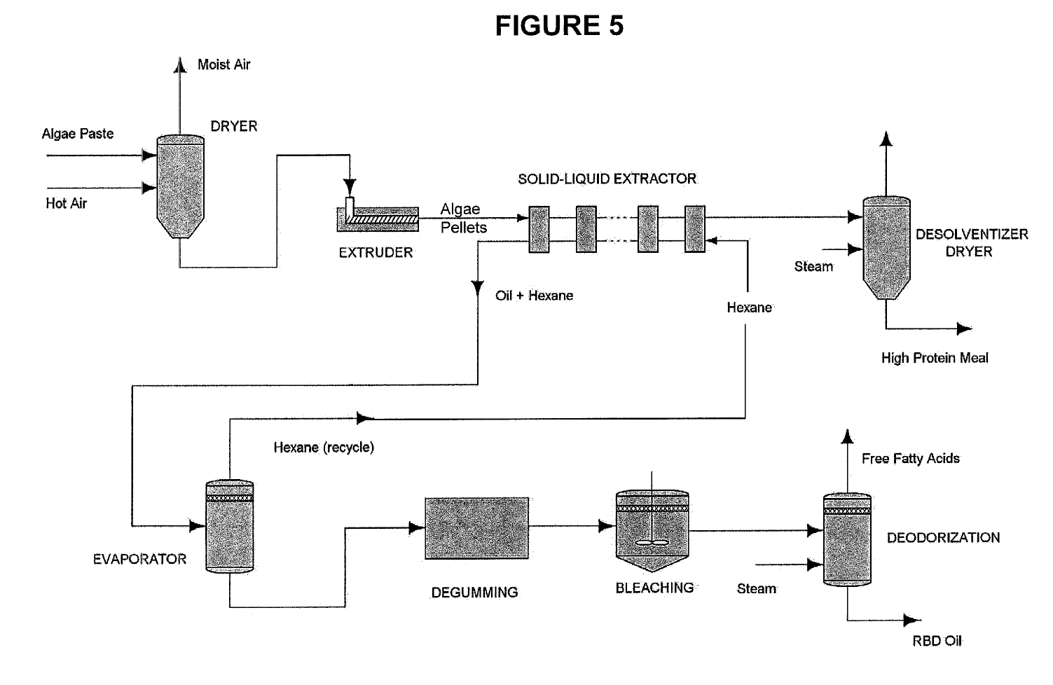

In other embodiments, the oil composition is extracted from a dried biomass, or dry extract, using counter-current contact with hexane, such as shown in FIG. 5 for algae comprising one or more of steps of: degumming, bleaching, and deodorizing for or oil extraction using hexane. The method is also described in Example 5. The oil composition within the dried biomass can be separated from the dry biomass using counter-current contact with hexane. The oil will dissolve into hexane, or other similar solvents, forming a solution called miscella. Cellular solids do not dissolve, and can be collected separately from the miscella. Most extractors utilize a conveyor system to draw the solids through the solvent solution, ensuring that the material is completely surrounded by miscella at all times. Solvent is usually pumped in the opposite direction of the conveyor. This countercurrent arrangement allows the extracted material to be discharged from one end of the machine while concentrated miscella (solvent and extractable) is taken from the other end. The solvent selected for extraction is generally less dense than the solids so that the powdery material left over after all the oil is extracted will stay on the conveyor, and not float on the surface of the miscella as it is collected. The concentrated miscella discharges from the extractor through a hydroclone, which scrubs fine particles from the oil/solvent mix before being pumped to the distillation system. Miscella from the extractor is then fed to a distillation process where algae oil is separated from the solvent. This allows recovery and reuse of the solvent, and purifies the oil to a point where it is ready for downstream processing. Distillation takes advantage of the difference in boiling points of the solvent and oil to separate the two components.

The next process for recovering oil from a dried extract is desolventizing. In one embodiment, material from the solvent extractor must be desolventized, then dried and cooled before it can be fed to an anaerobic or aerobic digester. This process is can be performed by a desolventiser-toaster, which consists of a vertical stack of several cylindrical gas-tight pans, each having a steam-heated bottom. Desolventizers generally have three sections: a pre-desolventizing section, a desolventizing section, and a toasting and stripping section. In the pre-desolventizing section, hexane is evaporated by indirect heating via heated trays. Solids continue to the desolventizing section, where most of the hexane is evaporated by condensing live steam. In the toasting and stripping section a combination of indirect and live steam is used to strip the remaining hexane while at the same time toasting the material. The solvent laden material enters the top of the desolventizer-toaster (DT) and lands on the steam heated pre-desolventizing tray(s) where it is evenly distributed by a sweep arm. The material flows from one tray to the next through tray openings. As it rises up through the material, the steam provides specific heat and a carrier gas to strip final traces of solvent from the material. The amount of live steam that is condensed is directly proportional to the amount of solvent in the material, for example, one kg of condensing water vapor evaporating between 6 and 7 kg of hexane.

In some embodiments, instead of harvesting the organism and then extracting the product, the products may be directly collected from the growth area and further processed by the processing/extraction unit (304). For example, hydrocarbons secreted from cells can be centrifuged to separate the hydrocarbons in a hydrophobic layer from contaminants in an aqueous layer and optionally from any solid materials. Material containing cell or cell fractions can be treated with proteases to degrade contaminating proteins before or after centrifugation. In some instances the contaminating proteins are associated, possibly covalently, to hydrocarbons or hydrocarbon precursors which form hydrocarbons upon removal of the protein. In other instances, the hydrocarbon molecules are in a preparation that also contains proteins. Proteases can be added to hydrocarbon preparations containing proteins to degrade proteins (for example, the commercially available protease from Streptomyces griseus can be used). After digestion, the hydrocarbons are can be purified from residual proteins, peptide fragments, and amino acids. This purification can be accomplished, for example, by methods such as centrifugation and filtration. Extracellular hydrocarbons can also be extracted in vivo from living organisms, such as from algae, which are then returned to the growth area by exposure of the cells to a non-toxic extraction solvent, followed by separation of the living cells and the hydrophobic fraction of extraction solvent and hydrocarbons, wherein the separated living cells are then returned to a growth area, such as an open growth area, or a closed one such as a stainless steel fermentor or photobioreactor (see for example, Biotechnol Bioeng. 2004 Dec. 5; 88(5):593-600; Biotechnol Bioeng. 2004 Mar. 5; 85(5):475-81).

In some embodiments, the extraction process uses approximately 0.30-50 MW. For example, in some embodiments, the extraction process uses less than approximately 50, 40, 35, 34, 30, 25, 24, 5.0, 1.0, 0.50, 0.40, 0.35, 0.30 MW. In some embodiments, between 2.0 to 800 MM Btu/hr (1 million British thermal units per hour) is used. For example, in some embodiments, the extraction process uses less than approximately 800, 770, 700, 500, 300, 190, 191, 15, 13, 12, 5, 3, or 2.8 Btu/hr.

The oil composition, or green crude, derived from the organism in the systems described herein can be between approximately 50-100,000 barrels per day (bpd). In some embodiments, between approximately 50-50,000, 50-40,000, 80-20,000, 80-15,000 or 80-10,000 bpd are produced. In some embodiments, the IBR produces at least approximately 50, 80, 90, 100, 200, 300, 350, 400, 500, 1000, 2000, 3000, 4000, 5000, 10,000, 15,000, 20,000, 25,000, 30,000, 40,000, or 50,000 bpd. The IBR can produce between approximately 5-1,000,000 MT/yr of oil composition derived from the microorganism. In some embodiments, the IBR produces between approximately 50-5,000,000, 100-500,000, 1000-50,000, or 1000-20,000 MT/yr of the oil composition. In some embodiments, the IBR produces at least approximately 80, 90, 100, 200, 300, 350, 400, 500, 1000, 2000, 3000, 4000, 5000, 10,000, 15,000, 20,000, or 500,000 MT/yr of oil composition.

### Refining Unit

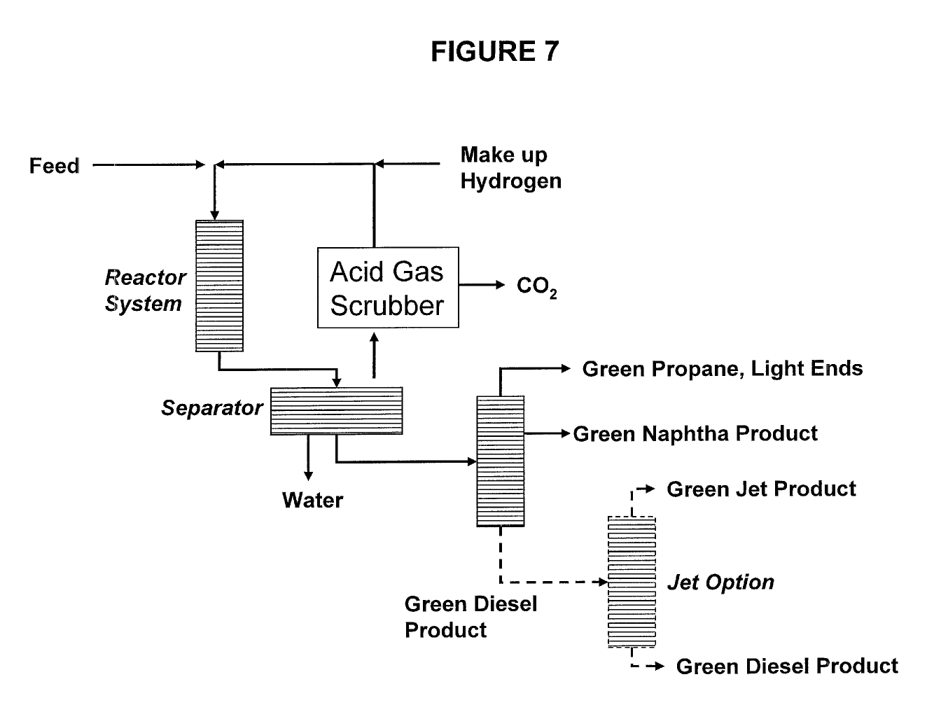

The oil composition is then transported to one or more refineries (110, FIG. 1). Transport may be by means such as by pipelines, trucks, rail or ships. The oil composition can be processed by a refining process to remove contaminants, such as heteroatoms, such as by hydrotreating. The oil compositions can be refined to produce to produce diesel fuel, jet fuel, or both (112). An example of this refining process is depicted in FIG. 7. The refining process can include, but not be limited to processes such described in US20090065395 or US20090065393, and can be commercially available refining processes such as Ecofining™ by UOP, or Bio-Synfining™ by Syntroleum. The refining process can generate one or more types of fuel products. For example, the refining process can produce one type of fuel product, such as diesel fuel, or can produce diesel fuel and jet fuel. In some embodiments, the refining process can be performed by a single unit that can have different modes, wherein one mode can be used to produce a single type of fuel product, such as diesel, or more than one fuel product, such as diesel and jet fuel.

The oil compositions can also be processed by a petroleum refinery to generate gasoline (114). The oil compositions can be subjected to more than one refining process. For example, the oil composition can be subjected to a first refining process, such as contaminant or heteroatom removal, prior to a second refining process such as cracking or isomerization. Various refining processes can be performed in conjunction with one or more other refining process to generate fuel products from the oil compositions. Furthermore, products from a first refining process can be used to in a second refining process. For example, a first refining process produces jet fuel and diesel, as well as light hydrocarbons and naphtha (112, FIG. 1, FIG. 7). The light hydrocarbons and naphtha can then be further refined to generate gasoline (114, FIG. 1). The various refining processes can be in a single unit of an IBR or separate units for an IBR. For example, a first refinery produces jet fuel and diesel, light hydrocarbons and naphtha. The light hydrocarbons and naphtha are then produced by a second refinery.

The refining of the oil compositions can generate, and use, hydrogen, (H₂). For example, hydroprocessing, such as hydrotreating, can require the use of hydrogen. In some embodiments, the IBR uses at least approximately 100,000, 150,00, 200,000, 220,000, 225,000, 230,000, 235,000, 240,000, 245,000, 250,000, 300,000, 400,000, 500,000, 600,000, 700,000, 725,000, 730,000, 735,000, 740,000, 745,000, 750,000, or 800,000 standard cubic feet per day (SCFD) of H₂. The can be used by the refining unit for producing diesel and jet fuel (112, FIG. 1). In some embodiments, the IBR comprises more than one refining unit, such as a petroleum refinery unit that generates H₂, and other refining unit that uses hydrotreating, such that the H₂ is used by the refining unit that is performing hydrotreatment. The H₂ can be transported by truck or pipeline.

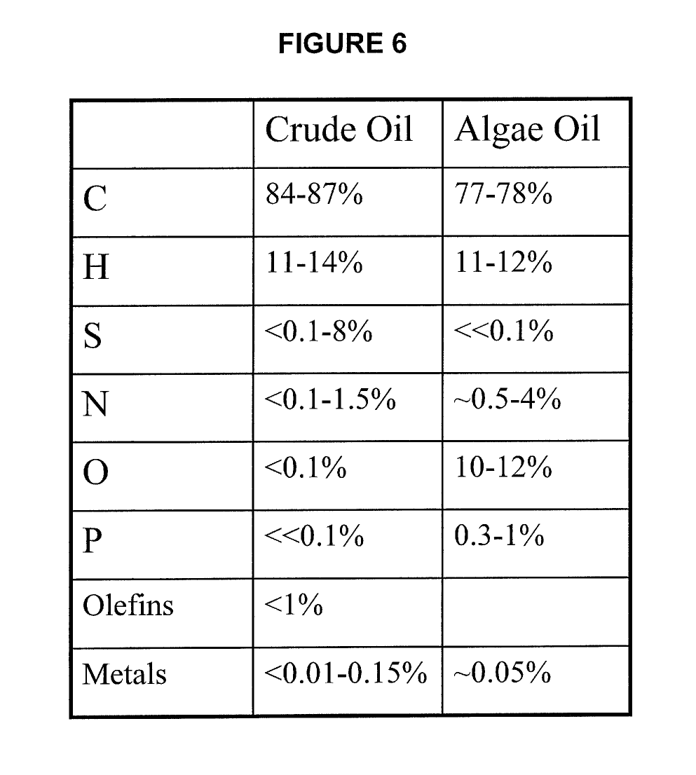

The oil composition harvested or extracted from an organism often contains contaminants and can be processed to remove these contaminants, by methods including, but not limited to, hydrotreating. For example, an oil composition derived from an organism contains larger amounts of heteroatoms than a fossil fuel oil composition (see for example, FIG. 6, comparing content of algal oils and crude oil from fossil fuels). An oil composition derived from algae can comprise significant levels (for example, 1% to greater than 40%) of a variety of other oil or lipid components including, but not limited to, chlorophylls and/or chlorophyllides, isoprenoids and carotenoids, and phospholipids. In an example, saline algae such as Duneliella viridis can deliver oils containing 30-40% of phospholipids and green algae deliver oils containing significant levels (for example, greater than 1% to much greater than 1% w/w) of chlorophylls or derivatives thereof. In some instances, an oil composition comprises hydrocarbons of the form of terpenes, isoprenoids, lipids, alkyl esters, alkaloids, or phenyl propanoids. Often an oil composition extracted from an organism contains biological molecules that contain heteroatoms such as chlorophyll, fatty acids, or phospholipids. Thus, the first refining process can include having the heteroatoms (for example, oxygen, nitrogen, phosphorous, sulfur, and metal) of an oil composition derived from an organism removed.

Heteroatoms can be removed by hydrogenolysis, a chemical reaction whereby a carbon-carbon or carbon-heteroatom single bond is cleaved or undergoes lysis by hydrogen. The heteroatom may vary, but examples include oxygen, nitrogen, or sulfur. A related reaction is hydrogenation, where hydrogen is added to the molecule, without cleaving bonds. Usually hydrogenolysis is conducted catalytically using hydrogen gas. In petroleum refineries, catalytic hydrogenolysis of feedstocks is conducted on a large scale to remove sulfur from feedstocks, releasing gaseous hydrogen sulfide (H₂S). The hydrogen sulfide is subsequently recovered in an amine treater and finally converted to elemental sulfur. Hydrogenolysis can be accompanied by hydrogenation. A hydrogenolysis reaction can be used to reduce the nitrogen content and is referred to hydrodenitrogenation (HDN). Many HDS units for desulfurizing naphthas within petroleum refineries are actually simultaneously denitrogenating to some extent as well. HDN can be performed as a method of removing nitrogen from an oil composition by creating ammonia or ammonium.

HDN can often require elevated temperatures (for example, approximately 300-500° C.) and high pressures of hydrogen (for example, greater than approximately 500 psi or even greater than approximately 1000 psi). In some instances, HDN can be carried out at a temperature of greater than approximately 100, 150, 200, 250, 300, 350, 400, 450, 500, 750, or 1000° C. In some instances, HDN can be carried out at a pressure of hydrogen of greater than approximately 100, 300, 500, 750, 1000, 1500, or 2000 psi. Exemplary HDN catalysts include those that comprise a support such as alumind (or aluminosilicates or silics) and can also comprise two or more metal compounds such as Co/Mo, Ni/Mo, W/Mo and the like.

In some instances, enzymes can be added to an oil composition to break up nitrogen containing compounds or molecules. Hydrogenation can be used to remove the nitrogen in the form of ammonia from an oil composition. Other methods of removing nitrogen from an oil composition have been developed and could be used with a system or method as described herein.

In an aspect, a chlorophyll or chlorophyllide can be removed from an oil composition to create a refined composition. Many types of oil compositions from photosynthetic organisms, such as algae, contain a significant portion of chlorophyll. Chlorophyll is often extracted with oil compositions from biomass as it is soluble in many of the solvents used for removing oil compositions. As provided herein, the methods and systems can remove much of the nitrogen and other heteroatoms associated with chlorophyll.

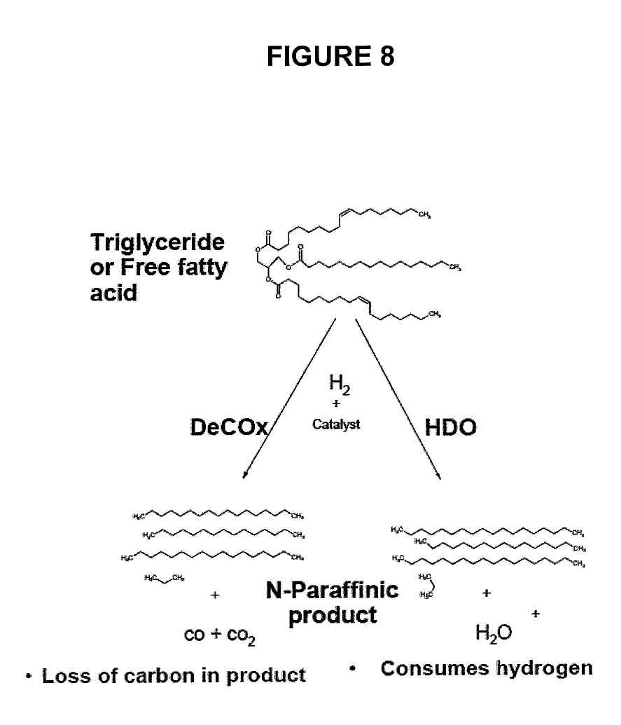

Another heteroatom that can be removed with a method or system herein is oxygen. An exemplary method of removing oxygen is hydrodeoxygenation (HDO). For example, triglycerides or fatty acids in the oil composition can be converted to N-paraffinic products (FIG. 8). The N-paraffinic products can then be used as diesel or jet fuel. The resulting product can be a mixture of H₂O, CO, and CO₂. Herein, HDO can refer to oxygen removal from a compound by means of hydrogen. Water is liberated in the reaction, and simultaneously olefinic double bonds are hydrogenated and any sulfur and nitrogen compounds can be removed as well. Reactions of the HDO step are exothermal. In the HDO step, exemplary catalysts containing a metal of the Group VIII and/or VIA of the periodic system of the elements can be used. The HDO catalyst can be a supported Pd, Pt, Ru, Rh, Ni, NiMo or CoMo catalyst, for example the support being activated carbon, alumina and/or silica. In some instances, any sulphur present can be removed during hydrodenitrogenation or hydrodeoxygenation as H₂S and can be collected in scrubbers.

HDO can often require elevated temperatures (for example, approximately 300-500° C.) and high pressures of hydrogen (for example, greater than approximately 500 psi or even greater than approximately 1000 psi). In some instances, HDO can be carried out at a temperature of greater than approximately 100, 150, 200, 250, 300, 350, 400, 450, 500, 750, or 1000° C. In some instances, HDO can be carried out at a pressure of hydrogen of greater than approximately 100, 300, 500, 750, 1000, 1500, or 2000 psi.

Other methods of removing oxygen from an oil composition include methods for removing oxygen from chemical compositions. Exemplary methods of removing oxygen include, but are not limited to, Barton-McCombie deoxygenation and the Wolff-Kishner reduction.

Examples hydrogenation catalysts include metals of Group VIb and/or Group VIII of the Periodic Table supported on a porous refractory oxide carrier. Examples of porous refractory oxides include alumina, silica, magnesia, silica-magnesia, zirconia, silica-zirconia, titania and silica-titania. In many instances, alumina or silica-alumina is used. Any conventional catalytically active ingredients for hydrogenation can be used as the active metal of a hydrogenation catalyst to be supported on the porous refractory oxide. For example, there can be used at least one member selected from the group consisting of metals (for example, chromium, molybdenum, tungsten) of Group VIb of the Periodic Table or the compounds of these metals and/or the metals (for example, iron, cobalt, nickel, platinum) of Group VIII of the Periodic Table or the compounds of these metals.

Metal contaminants can also be removed. For example, metals or metalloids can be removed by absorption of the metal or metalloids onto the surface of a catalyst. In some instances, hydrodemetallization (HDM) in which metals (for example, Mg and Na) and metalloids (for example, P) can be removed by absorption onto a catalyst. For example, the catalysts as described herein can comprise a support such as alumind (or aluminosilicates or silics) and can also comprise two or more metal compounds such as Co/Mo, Ni/Mo, W/Mo and the like. The steps of removing metal or metalloids can often require elevated temperatures (for example, approximately 300-500° C.) and high pressures of hydrogen (for example, greater than approximately 500 psi or even greater than approximately 1000 psi). In some instances, a metal removing step can be carried out at a temperature of greater than approximately 100, 150, 200, 250, 300, 350, 400, 450, 500, 750, or 1000° C. In some instances, a metal removing step can be carried out at a pressure of hydrogen of greater than approximately 100, 300, 500, 750, 1000, 1500, or 2000 psi.

Another method for removing metal from an oil composition is the Demet Process which can remove metals such as nickel and vanadium from a spent catalyst. The nickel and vanadium are converted to chlorides which are then washed out of the catalyst. Another exemplary method of removing metals is metal passivation, wherein materials can be used as additives which can be impregnated in a catalyst or added to the oil composition in the form of metal-organic compounds. Such materials can react with the metal contaminants and passivate the contaminants by forming less harmful compounds that remain on the catalyst. For example, antimony and bismuth are effective in passivating nickel and tin is effective in passivating vanadium. A number of proprietary passivation processes are available.

In some instances, the metal-removing catalyst comprises: a support of alumind, aluminosilicate, aluminosilic; and Co/Mo, Ni/Mo, or W/Mo. Exemplary supports of a metal-removing (for example, hydrodemetallizing) catalyst for a hydrocarbon oil herein include, but are not limited to, alumina, silica, silica-alumina, titania, magnesia and silica-magnesia can be used without any particular limitation as the support. Hydrodemetallization can be carried out in a fixed bed system. A reactor of a system here can be either a single-stage or a multistage reactor.

Catalysts described herein can be prepared by conventional methods. The alumina carrier can be prepared by neutralizing an acidic aluminum salt such as aluminum sulfate or aluminum nitrate with a base such as ammonia, or neutralizing an aluminate such as sodium aluminate with an acidic aluminum salt or an acid, washing the resulting gel and carrying out conventional treatments such as heating, aging, molding, drying and calcining.

In some instances, the system for removing contaminants further comprises a distilling device in fluidic communication configured to remove light hydrocarbons. Exemplary light hydrocarbons include, but are not limited to, methane, ethane, propane, butane, or C4 hydrocarbons or smaller, which can be subjected to a second refining process. The second refining process can be at a standard petroleum refinery (114) that produces gasoline.

The petroleum refinery (114) can perform refining processes that optimize the types, shapes, and sizes of the hydrocarbon mixture to produce a fuel product. Typical refining processes in the fuel industry include, but are not limited to, distillation, fractionation, extraction, solvent extraction, hydroprocessing, isomerization, dimerization, alkylation, and cracking. A cracking process typically refers to the process that breaks down hydrocarbons into smaller hydrocarbons, for example, by scission of a carbon-carbon bond. Complex organic molecules such as isoprenoids or heavy hydrocarbons can be cracked into simpler molecules (for example light hydrocarbons) by the breaking of carbon-carbon bonds in the precursors. Cracking is commonly performed by using high temperatures, catalysts, or a combination thereof. Examples of cracking methods include, but are not limited to, thermal cracking, fluid catalytic cracking, thermal catalytic cracking, catalytic cracking, steam cracking, and hydrocracking.

Catalytic and thermal cracking methods are routinely used in hydrocarbon and triglyceride oil processing. Catalytic methods involve the use of a catalyst, such as a solid acid catalyst. The catalyst can be silica-alumina or a zeolite, which result in the heterolytic, or asymmetric, breakage of a carbon-carbon bond to result in a carbocation and a hydride anion. These reactive intermediates then undergo either rearrangement or hydride transfer with another hydrocarbon. The reactions can thus regenerate the intermediates to result in a self-propagating chain mechanism. Hydrocarbons can also be processed to reduce, optionally to zero, the number of carbon-carbon double, or triple, bonds therein. Hydrocarbons can also be processed to remove or eliminate a ring or cyclic structure therein. Hydrocarbons can also be processed to increase the hydrogen:carbon ratio. This can include the addition of hydrogen (“hydrogenation”) and/or the “cracking” of hydrocarbons into smaller hydrocarbons.

Thermal methods involve the use of elevated temperature and pressure to reduce hydrocarbon size. An elevated temperature of about 800° C. and pressure of about 700 kPa can be used. These conditions generate “light,” a term that is sometimes used to refer to hydrogen-rich hydrocarbon molecules (as distinguished from photon flux), while also generating, by condensation, heavier hydrocarbon molecules which are relatively depleted of hydrogen. The methodology provides homolytic, or symmetrical, breakage and produces alkenes, which may be optionally enzymatically saturated as described above.

Refining the oil compositions disclosed herein can include generating diesel fuel by transesterification of triglycerides (TAG) contained in the oil composition derived from the organism. Transesterification reactions such as base catalyzed transesterification and transesterification using recombinant lipases can be used. For example, in a base-catalyzed transesterification process, the triacylglycerides are reacted with an alcohol, such as methanol or ethanol, in the presence of an alkaline catalyst, typically potassium hydroxide. This reaction forms methyl or ethyl esters and glycerin (glycerol) as a byproduct. Transesterification can also be performed by using an enzyme, such as a lipase instead of a base. Lipase-catalyzed transesterification can be carried out, for example, at a temperature between the room temperature and 80° C., and a mole ratio of the TAG to the lower alcohol of greater than 1:1, preferably about 3:1. Examples of lipases useful for transesterification can be found in, e.g. U.S. Pat. Nos. 4,798,793; 4,940,845; 5,156,963; 5,342,768; 5,776,741; 2009/0047721 and WO89/01032.

Diesel fuel can also be generated by cracking the oil composition in conjunction with hydrotreating to reduce carbon chain length and saturate double bonds, respectively. The material is then isomerized, also in conjunction with hydrotreating. The naphtha fraction can then be removed through distillation, followed by additional distillation to vaporize and distill components desired in the diesel fuel to meet a D975 standard (see below) while leaving components that are heavier than desired for meeting a D 975 standard.

In another embodiment for producing diesel, the first step of treating a triglyceride is hydroprocessing to saturate double bonds, followed by deoxygenation at elevated temperature in the presence of hydrogen and a catalyst. In some methods, hydrogenation and deoxygenation occur in the same reaction. In other methods deoxygenation occurs before hydrogenation. Isomerization is then optionally performed, also in the presence of hydrogen and a catalyst. Naphtha components are preferably removed through distillation. For examples, see U.S. Pat. No. 5,475,160 (hydrogenation of triglycerides); U.S. Pat. No. 5,091,116 (deoxygenation, hydrogenation and gas removal); U.S. Pat. No. 6,391,815 (hydrogenation); and U.S. Pat. No. 5,888,947 (isomerization).

A traditional ultra-low sulfur diesel can also be produced by the systems disclosed herein. First, the biomass can be converted to a syngas, a gaseous mixture rich in hydrogen and carbon monoxide. Then, the syngas is catalytically converted to liquids. Typically, the production of liquids is accomplished using Fischer-Tropsch (FT) synthesis. This technology applies to coal, natural gas, and heavy oils. In another embodiment, treating the oil composition to produce an alkane is performed by indirect liquefaction of the lipid composition.

Another refining method the systems disclosed herein can use is fluid catalytic cracking (FCC), which can be used to produce olefins, especially propylene from heavy crude fractions, which can be useful as jet fuel. The oil compositions produced herein can be converted to olefins. The process involves flowing the composition through an FCC zone and collecting a product stream comprised of olefins, which is useful as a jet fuel. The oil composition is contacted with a cracking catalyst at cracking conditions to provide a product stream comprising olefins and hydrocarbons useful as jet fuel. For example, the oil composition can be flowed through a FCC zone, which may comprise contacting the oil composition with a cracking catalyst at cracking conditions to provide a product stream comprising C2-C5 olefins.

For isomerization during refining, suitable isomerization catalysts that may be used include those that contain a molecular sieve and/or a metal from Group VII and/or a carrier. Preferably, the isomerization catalyst contains SAPO-11 or SAPO41 or ZSM-22 or ZSM-23 or ferrierite and Pt, Pd or Ni and Al₂O₃ or SiO₂. Typical isomerization catalysts are, for example, Pt/SAPO-11/Al₂O₃, Pt/ZSM-22/Al₂O₃, Pt/ZSM-23/Al₂O₃ and Pt/SAPO-11/SiO₂.

The system disclosed herein can produce one or more fuel compositions, such as diesel fuel, jet fuel, gasoline, or any combination thereof. The amount produced can be between approximately 1-100,000 barrels per day (bpd). In some embodiments, between approximately 50-100,000, 50-50,000, 80-25,000, 80-20,000 or 80-10,000 bpd are produced. In some embodiments, the IBR produces at least approximately 50, 80, 90, 100, 200, 300, 350, 400, 500, 1000, 2000, 3000, 4000, 5000, 10,000, 15,000, 20,000, 25,000, 30,000, 40,000, or 50,000 bpd. The IBR can produce between approximately 1-1,000,000 MT/yr of fuel compositions. In some embodiments, the IBR produces between approximately 5-5,000,000, 10-500,000, 500-50,000, or 1000-20,000 MT/yr of the oil composition. In some embodiments, the IBR produces at least approximately 1, 5, 10, 100, 500, 1000, 2000, 3000, 4000, 5000, 10,000, 15,000, 20,000, 100,000, 150,000, 200,000, 250,000, 300,000, 400,000, 450,000 or 500,000 MT/yr of a fuel product.

In some embodiments, the fuel produced is diesel and jet fuel. In other embodiments, the fuel produced is diesel fuel alone. In other embodiments, the fuel product is jet fuel. For example, the IBR can produce between approximately 1-100,000 barrels per day (bpd) of jet fuel and diesel fuel. In some embodiments, between approximately 50-100,000, 50-50,000, 80-20,000, 80-10,000 or 80-9,000 bpd of jet fuel and diesel is produced. In some embodiments, the IBR produces at least approximately 50, 80, 90, 100, 200, 300, 350, 400, 500, 1000, 2000, 3000, 4000, 5000, 6,000, 7000, 8000, 9000, 10000, 12000, 15000, 20000, 25000, 30000, 40000, or 50000 bpd of jet fuel and diesel fuel. For example, in some embodiments, at least approximately 30 bpd of jet fuel and at least approximately 50 bpd of diesel is produced. In other embodiments, at least approximately 4,000 bpd of jet fuel and approximately at least 5,000 bpd of diesel is produced.

In some embodiments, between approximately 10-50% of the fuel product produced by the IBR is jet fuel. In other embodiments, at least approximately 10%, 15%, 20%, 25%, 30%, 35%, 40%, 45%, or 50% of the fuel product is jet fuel. In other embodiments, at least approximately 10%, 15%, 20%, 25%, 30%, 35%, 40%, 45%, 50%, 60%, 70%, 75%, 80%, 90%, 95%, 99%, or 100% of the fuel product is diesel. The refining process, or process for generating the fuel product, can use between 0.10 to 20, 0.1-15, or 0.1-10 MW. For example, the process may use less than approximately 10 MW, or at least approximately 0.1 MW.

### Fuel Compositions

The IBR produces oil compositions that can be used to produce fuel compositions. The fuel compositions can be precursors or products conventionally derived from crude oil, or petroleum, such as, but not limited to, liquid petroleum gas, naphtha (ligroin), gasoline, kerosene, diesel, lubricating oil, heavy gas, coke, asphalt, tar, and waxes. For example, fuel products may include small alkanes (for example, 1 to approximately 4 carbons) such as methane, ethane, propane, or butane, which may be used for heating (such as in cooking) or making plastics. Fuel products may also include molecules with a carbon backbone of approximately 5 to approximately 9 carbon atoms, such as naphtha or ligroin, or their precursors. Other fuel products may be about 5 to about 12 carbon atoms or cycloalkanes used as gasoline or motor fuel. Molecules and aromatics of approximately 10 to approximately 18 carbons, such as kerosene, or its precursors, may also be fuel products. Fuel products may also include molecules, or their precursors, with more than 12 carbons, such as used for lubricating oil. Other fuel products include heavy gas or fuel oil, or their precursors, typically containing alkanes, cycloalkanes, and aromatics of approximately 20 to approximately 70 carbons. Fuel products also includes other residuals from crude oil, such as coke, asphalt, tar, and waxes, generally containing multiple rings with about 70 or more carbons, and their precursors.

In some embodiments, the IBR produces alcohols, such as ethanol, for example from the solid extract processing units as described herein, which can be used for generating a fuel composition disclosed herein.

The fuel compositions produced by the IBR can be suitable for transportation fuels, such as diesel fuel, jet fuel, and gasoline. Traditional diesel fuels are petroleum distillates rich in paraffinic hydrocarbons. They have boiling ranges as broad as 370° F. to 780° F., which are suitable for combustion in a compression ignition engine, such as a diesel engine vehicle. The American Society of Testing and Materials (ASTM) establishes the grade of diesel according to the boiling range, along with allowable ranges of other fuel properties, such as cetane number, cloud point, flash point, viscosity, aniline point, sulfur content, water content, ash content, copper strip corrosion, and carbon residue. Technically, any hydrocarbon distillate material derived from biomass or otherwise that meets the appropriate ASTM specification can be defined as diesel fuel (ASTM D975), jet fuel (ASTM D1655), or as biodiesel (ASTM D6751).

Thus, after extraction, lipid and/or hydrocarbon components recovered from the microbial biomass described herein can be subjected to refining as described herein to manufacture a fuel for use in diesel vehicles and jet engines.

Typically, biodiesel comprises C14-C18 alkyl esters. Various processes convert biomass or a lipid produced and isolated as described herein to diesel fuels. A preferred method to produce biodiesel is by transesterification of a lipid as described herein. A preferred alkyl ester for use as biodiesel is a methyl ester or ethyl ester.

Biodiesel produced by a method described herein can be used alone or blended with conventional diesel fuel at any concentration in most modern diesel-engine vehicles. When blended with conventional diesel fuel (petroleum diesel), biodiesel may be present from about 0.1% to about 99.9%. Much of the world uses a system known as the “B” factor to state the amount of biodiesel in any fuel mix. For example, fuel containing 20% biodiesel is labeled B20. Pure biodiesel is referred to as B100.

Biodiesel can also be used as a heating fuel in domestic and commercial boilers. Existing oil boilers may contain rubber parts and may require conversion to run on biodiesel. The conversion process is usually relatively simple, involving the exchange of rubber parts for synthetic parts due to biodiesel being a strong solvent. Due to its strong solvent power, burning biodiesel will increase the efficiency of boilers.

Biodiesel can be used as an additive in formulations of diesel to increase the lubricity of pure Ultra-Low Sulfur Diesel (ULSD) fuel, which is advantageous because it has virtually no sulfur content. Biodiesel is typically a better solvent than petrodiesel and can be used to break down deposits of residues in the fuel lines of vehicles that have previously been run on petrodiesel.

The IBR can also produce jet fuel. The most common jet fuel is an unleaded/paraffin oil-based fuel classified as Aeroplane A-1, which is produced to an internationally standardized set of specifications. Aeroplane fuel is typically a mixture of a large number of different hydrocarbons, possibly as many as a thousand or more. The range of their sizes (molecular weights or carbon numbers) is restricted by the requirements for the product, for example, freezing point or smoke point. Kerosene-type Aeroplane fuel (including Jet A and Jet A-1) has a carbon number distribution between about 8 and 16 carbon numbers. Wide-cut or naphtha-type Aeroplane fuel (including Jet B) typically has a carbon number distribution between about 5 and 15 carbons.

Often, oil compositions derived from biomass are suitable for producing high-octane hydrocarbon products. Thus, one embodiment describes a method of forming a fuel product comprising: forming one or more light hydrocarbons having 4 to 12 carbons having an Octane number of 80 or higher by cracking a biomass feedstock, and blending the one or more light hydrocarbons with the Octane number of 80 or higher with a hydrocarbon having an Octane number of 80 or less. Typically, the hydrocarbons having an Octane number of 80 or less are fossil fuels derived from refining crude oil.

The fuel compositions may also be blended or combined into mixtures to obtain an end product. For example, the fuel products may be blended to form gasoline of various grades, gasoline with or without additives, lubricating oils of various weights and grades, kerosene of various grades, jet fuel, diesel fuel, heating oil, and chemicals for making plastics and other polymers. Compositions of the fuel products described herein may be combined or blended with fuel products produced by other means.

In some instances, a product (such as a fuel product) contemplated herein comprises one or more carbons derived from an inorganic carbon source. In an embodiment, at least 10%, 20%, 30%, 40%, 50%, 60%, 70%, 80%, 90%, 95%, or 99% of the carbons of a product as described herein are derived from an inorganic carbon source. Examples of inorganic carbon sources include, but are not limited to, carbon dioxide, carbonate, bicarbonate, and carbonic acid. The product can be an organic molecule with carbons from an inorganic carbon source that were fixed during photosynthesis.

A product herein can be described by its Carbon Isotope Distribution (CID). At the molecular level, CID is the statistical likelihood of a single carbon atom within a molecule to be one of the naturally occurring carbon isotopes (for example, ¹²C, ¹³C, or ¹⁴C). At the bulk level of a product, CID may be the relative abundance of naturally occurring carbon isotopes (for example, ¹²C, ¹³C, or ¹⁴C) in a compound containing at least one carbon atom. While it is noted that CID of each fossil fuel may differ based on its source, CID(fos) (for example, CID of carbon in a fossil fuel, for example, petroleum, natural gas, and coal) is distinguishable from CID(atm) (for example, the CID of carbon in current atmospheric carbon dioxide). Additionally, CID(photo-atm) refers to the CID of a carbon-based compound made by photosynthesis in recent history where the source of inorganic carbon was carbon dioxide in the atmosphere. CID(photo-fos) refers to the CID of a carbon based compound made by photosynthesis in recent history where the source of substantially all of the inorganic carbon was carbon dioxide produced by the burning of fossil fuels (for example, coal, natural gas, and/or petroleum).

The exact distribution is also a characteristic of 1) the type of photosynthetic organism that produced the molecule and 2) the source of inorganic carbon. These isotope distributions can be used to define the composition of photosynthetically-derived fuel products.

Carbon isotopes are unevenly distributed among and within different compounds and the isotopic distribution can reveal information about the physical, chemical, and metabolic processes involved in carbon transformations. The overall abundance of ¹³C relative to ¹²C in photosynthetic organism tissue is commonly less than in the carbon of atmospheric carbon dioxide, indicating that carbon isotope discrimination occurs in the incorporation of carbon dioxide into photosynthetic biomass.

Some fuel products can be produced from biomass, sometimes after refining, and the products are identical to existing petrochemicals. Some of the fuel products may not be the same as existing petrochemicals. In an embodiment, a fuel product or composition is identical to an existing petrochemical, except for the carbon isotope distribution. For example, it is believed no fossil fuel petrochemicals have a δ¹³C distribution of less than −32‰, whereas fuel products as described herein can have a δ¹³C distribution of less than −32‰, −35‰, −40‰, −45‰, −50‰, −55‰, or −60‰. In another embodiment, a fuel product or composition is similar but not the same as an existing fossil fuel petrochemical and has δ¹³C distribution of less than −32‰, −35‰, −40‰, −45‰, −50‰, −55‰, or −60. However, although a molecule may not exist in conventional petrochemicals or refining, it may still be useful in these industries. For example, a hydrocarbon can be produced that is in the boiling point range of gasoline, and that could be used as gasoline or an additive, even though the hydrocarbon does not normally occur in gasoline. A fuel product can be a composition comprising: hydrogen and carbon molecules, wherein the hydrogen and carbon molecules are at least approximately 65% of the atomic weight of the composition, and wherein the δ¹³C distribution of the composition is less than −32‰. In other embodiments, the fuel product can be a composition comprising: hydrogen and carbon molecules, wherein the hydrogen and carbon molecules are at least 80% of the atomic weight of the composition, and wherein the δ¹³C distribution of the composition is less than −32‰. For some fuel products described herein, the hydrogen and carbon molecules are at least 90% of the atomic weight of the composition. For example, a biodiesel or fatty acid methyl ester (which have less than 90% hydrogen and carbon molecules by weight) may not be part of the composition. In still other compositions, the hydrogen and carbon molecules are at least 95 or 99% of the atomic weight of the composition. In yet other compositions, the hydrogen and carbon molecules are 100% of the atomic weight of the composition. In some instances, the composition is a liquid. In other instances, the composition is a fuel additive or a fuel product.

Also described herein is a fuel composition comprising hydrogen and carbon molecules, wherein the hydrogen and carbon molecules are at least 65%, or at least 80% of the atomic weight of the composition, and wherein the δ¹³C distribution of the composition is less than −32‰ and a fuel component. In some embodiments, the δ¹³C distribution of the composition is less than about −35‰, −40‰, −45‰, −50‰, −55‰, or −60‰. In some instances, the fuel component is a blending fuel which may be fossil fuel, gasoline, diesel, ethanol, jet fuel, or any combination thereof. In still other instances, the blending fuel has a δ¹³C distribution of greater than −32‰. For some fuel products described herein, the fuel component is a fuel additive which may be MTBE, an anti-oxidant, an antistatic agent, a corrosion inhibitor, and any combination thereof. A fuel product as described herein may be a product produced by blending a fuel product as described and a fuel component. In some instances, the fuel product has a δ¹³C distribution of greater than −32‰. In other instances, the fuel product has a δ¹³C distribution of less than −32‰. For example, a composition extracted from an organism can be blended with a fuel component prior to refining (for example, cracking) in order to produce a fuel product as described herein. A fuel component, as described, can be a fossil fuel, or a mixing blend for generating a fuel product. For example, a mixture for fuel blending may be a hydrocarbon mixture that is suitable for blending with another hydrocarbon mixture to produce a fuel product. For example, a mixture of light alkanes may not have a certain octane number to be suitable for a type of fuel, however, it can be blended with a high octane mixture to produce a fuel product. In another example, a composition with a δ¹³C distribution of less than −32‰ is blended with a hydrocarbon mixture for fuel blending to create a fuel product.

The carbon isotopes can also be used to trace the biomass feedstock to which a fuel composition was produced from. The isotope can be introduced into a biomass hydrocarbon in the course of its biosynthesis. The carbon isotopes can also be used to trace the biomass feedstock to which a product produced from processing of the solid extracts. For example, carbon isotopes can be used to trace the alcohols, such as ethanol, produced. In some embodiments, the carbon isotopes can be used to trace terpenes or isoprenoids. The carbon isotopes can then serve as markers in the hydrocarbon feedstocks, or other products, such as alcohols (e.g. ethanol), produced. The tagged hydrocarbon feedstocks can be subjected to the refining processes described herein to produce light hydrocarbon products tagged with carbon isotopes. The isotopes allows for the identification of the tagged products, either alone or in combination with other untagged products, such that the tagged products can be traced back to their biomass feedstocks.

### Additional Processing Units

The IBR can also comprise additional processing units, such as for the processing of non-oil compositions resulting from the processing and extraction of an oil composition from the organism (204, FIG. 2). For example, the IBR can comprise a processing unit (312, FIG. 3), or waste processing unit, for the solid extracts derived from the organism. The solid extracts, such as algal solids, can be processed by the additional processing units to generate animal feed, human feed, biofuel, or methane. The additional processing module can comprise anaerobic digestion, such as methods known in the arts (e.g. WO 03/042117, US20020079266, US20080311640), which can be used to produce methane (410, FIG. 4), that can be burned to heat water or generate electricity. The anaerobic digestion can also generate nutrients and CO₂ that are inputted back into the production field. The solid extracts can also be processed by fermentation by methods known in the arts, (e.g. US20090006280) to produce alcohol, including but not limited to methanol, ethanol, propanol, and butanol, as well as gaseous co-products such as carbon dioxide. Alternatively, the processing module can be a food plant to process the organism solids to animal feed or human feed.

For example, the dried biomass from desolventizing, or from the extraction process, can be fed to an anaerobic digester, where the biomass is converted to a gas stream containing methane. The gas stream and residual nutrients can then be recycled to the production unit for reuse. Dried biomass left over after digestion could be sold as animal feed. The processing unit can also be a plant that processes the solid extracts to other human products, such as human feed and cosmetic products. The processing unit for the solid extracts, or “waste,” can use approximately 0.03 MW to 5 MW, in some embodiments, the processing unit uses less than approximately 5, 4, 3, 2, 1, 0.5, 0.25, 0.10, 0.05, 0.03 MW. The processing unit may use between −6.0 to −400 MM Btu/hr, such as less than −400, −370, −368, −6.0, −5.4 MM Btu/hr. The processing unit may generate power or energy to operate the IBR. For example, in some embodiments, at least approximately 5, 10, 15, 20, 25, 30, 35, 40, 45, 50, 55, 60, 65, 70, 75, 80, 85, or 90, 95, 99, or 100% of the power required to run the IBR by generated by the processing unit for the solids.

The additional processing units can also produce products, or byproducts that are inputted into other units of the IBR. For example, one unit can generate CO₂ (410, FIG. 4) that is be used by the organism in the growth area (402, FIG. 4). Byproducts of a processing unit for the solid extracts area (312, FIG. 3; 410, FIG. 4) can also generate nutrients that are inputted into the production field (302, FIG. 3; 402, FIG. 4), such as water, CO₂, phosphates, nitrates, sulfites, and other nutrients and minerals.

### Carbon Credits

The IBR uses carbon dioxide in its growth area to maintain and grow organisms for producing fuel compositions. As a result, the IBR can sequester CO₂ and be used as a basis for earning “carbon credits.” Carbon sequestered in carbon sinks, such as the production field of the IBR, can be the basis for earning “carbon credits” that can be traded as part of an emissions trading scheme. Emission trading schemes typically utilize a cap-and-trade arrangement wherein a governing body sets a cap on allowable emissions and issues emission permits that represent the right to emit a specific amount of a pollutant. Participants that do not have enough emission permits to cover their emissions can purchase credits from participants that have extra permits. The carbon credits may also be used as a tax credit, for example the entity operating or who owns the IBR, or modules of the IBR may be able to use the carbon credits to offset their tax liability.

Participants are also able to purchase credits from entities that have earned credits by creating a net reduction in greenhouse gases. The Kyoto Protocol to the United Nations Framework Convention on Climate Change (an international environmental treaty aimed at reducing emissions of greenhouse gases) has established a framework for emissions trading schemes. The European Union Emission Trading Scheme, which is modeled on the Kyoto Protocol, is the largest emissions trading scheme. Various emissions trading schemes have also been in use within the United States for some time.

Carbon credits are typically awarded to entities that have produced a verifiable reduction in atmospheric carbon. In addition to being traded under an emissions trading scheme, carbon credits can be purchased by companies or individuals who wish to lower their carbon footprint on a voluntary basis (i.e., outside of an emissions trading scheme). Carbon credits typically must be validated or certified, usually by a governing body, before they can be traded meaningfully in a marketplace. For instance, the Kyoto Protocol has established the Clean Development Mechanism (CDM), which validates and measures projects to ensure they produce authentic benefits to the environment.

The IBR can therefore be used to profit through selling fuel compositions, as well as by selling carbon credits, or using the carbon credits to reduce their taxes. The IBR can also generate profits from selling the oil composition to external entities that can refine and sell the resulting fuel compositions. The IBR can also sell the products generated by processing the solid extracts, such as animal feed, human feed, cosmetics, biofuels, and power. The IBR can also see the solid extracts themselves to external entitles, which can process and sell the resulting products.

While preferred embodiments of the present invention have been shown and described herein, it will be obvious to those skilled in the art that such embodiments are provided by way of example only. Numerous variations, changes, and substitutions will now occur to those skilled in the art without departing from the present invention. It should be understood that various alternatives to the embodiments of the present invention described herein may be employed in practicing the present invention. It is intended that the following claims define the scope of the present invention and that methods and structures within the scope of these claims and their equivalents be covered thereby.

### EXAMPLES

### Example 1

### System for Producing Fuels from Developed Microorganism

FIG. 1 illustrates an exemplary method and system as described herein. In 102, a strain of algal is developed to have an increased ability to produce a fatty acid as compared to an unmodified strain. For example, a strain of Chlamydomonas reinhardtii is genetically modified by inserting a vector that expresses thioesterase into its chloroplast genome. The modification results in the genetically modified strain producing a naturally occurring fatty acid at levels greater than an unmodified strain.

The modified strain is then grown in an open race way pond of approximately 100 acres in 104. The algae strain is then harvested (106) and the oil composition comprising the increased levels of fatty acids as compared to an oil composition from an unmodified strain is recovered (108). The oil composition is then transported by a pipeline (110) to a refinery for hydrotreating (112), such as the UOP Ecofining™ system. Jet fuel and diesel fuel are produced by the refinery (112). Naphtha and light hydrocarbons produced by the first refinery (112) is then refined by a second refinery, a petroleum refinery (114), to produce gasoline.

### Example 2

### IBR with Petroleum Refinery Unit and Industrial CO₂ Source

FIG. 3 illustrates an exemplary method and system of an IBR. In 302, an algal strain is grown in a production field of approximately 1000 acres of non-arable land. Approximately 20,000 acre-ft/yr of brackish water is irrigated into the production field. Approximately 150,000 MT/yr of CO₂ generated by a petroleum refinery (308) and/or an industrial plant (310), such as a manufacturing plant, is inputted into the production field. The algae is harvested and processed to extract and separate algal oils from algal solids (304) by, for example, using hexane extraction. Approximately 200-350 bpd of algal oils and approximately 110 MT/day of algal solids are produced.

Byproducts of the processing and extraction of the algal oils and algal solids, such as water and salts, are inputted back into the production field (302). The algal oils are transported to a refinery for hydrotreating, by a process such as Ecofining™ (306). Hydrogen is supplied for the Ecofining™ by a petroleum refinery (308) that inputs approximately 735,000 SCFD of hydrogen into the refinery for hydrotreating the oil composition (such as an Ecofining™ refinery). The hydrotreating refinery produces approximately 330 bpd of diesel fuel, jet fuel or both. Products from the such as light hydrocarbons and naphtha that result from the refining of the oil composition can then be refined by the petroleum refinery (308) of the IBR. The algal oils not refined by the refinery for hydrotreating (306) can be directly refined by the petroleum refinery (308). The petroleum refinery produces gasoline and olefins.

The algal solids are processed to generate biofuels, power, and animal feed (312). Processing of the algal solids produces byproducts such as nutrients that are inputted into the production field (302).

### Example 3

### IBR Using Atmospheric/Anthropogenic CO₂

FIG. 4 illustrates an exemplary method and system of an IBR. In 402, an algal strain is grown in a production field of approximately 350 acres of non-arable land. Approximately 20,000 acre-ft/yr of brackish water is irrigated into the production field. Approximately 65,000 MT/yr of CO₂ from the atmosphere (412) is inputted into the field. The algae is harvested and processed to extract and separate algal oils from algal solids (404) by using hexane extraction. Approximately 80 bpd of algal oils and approximately 44 MT/day of algal solids are produced.

Byproducts of the processing and extraction of the algal oils and algal solids, such as water and salts, are inputted back into the production field. The algal oils are then transported to a refinery (406). A hydrogen pipeline (408) inputs approximately 245;000 SCFD of hydrogen into the refinery, where the refinery produces approximately 60 bpd of diesel fuel, jet fuel or both. The algal solids are processed by anaerobic digestion (410) producing methane that can be used to generate power, such as heat.

### Example 4

### IBR Using Wet Extraction Process

In this example, a demonstration facility and a second, larger facility, commercial scale, are built. The parameters of the facilities are shown in Table 1. Both facilities utilize substantially identical processes to convert oils extracted from algae into liquid transportation fuels.

TABLE 1
Design Parameters for Demonstration and Commercial Scale Facilities.

| | Facility: | | | |
|---|---|---|---|---|
| | Demonstration | | | |
| | Scale | | Commercial Scale | |
| Algae Pond Acreage: | 300 | | 20,400 | |
| CO2 Capture (MT/day): | 56 | | 4,878 | |
| CO2 Utilization: | 60% | | 90% | |
| Extractable Liquid Fraction: | 50% | | 60% | |
| Refinable Liquid Fraction: | 85% | | 90% | |
| Refined Oil (bbl/day)¹ | 106 | | 10,000 | |
| Refinery Output Mode:² | Mixed | Diesel | Mixed | Diesel |
| Green Diesel (bbl/day): | 60 | 95 | 5,000 | 9,000 |
| Green Jet (bbl/day): | 37 | 0 | 4,300 | 0 |

¹Values are for mean productivity of algae ponds.
²Refining process to generate fuels can be run in two different modes: 1) Mixed Mode, where output is a mixture of green jet and green diesel, and 2) Diesel Mode, where output is primarily green diesel.

### Production Unit

One or more algal strains are developed to have an improved ability to produce oils. The algae strain is grown in a demonstration-scale facility, which utilizes approximately 500 acres. The refining process of the green crude is performed at a different site. The commercial-scale facility utilizes approximately 22,000 acres, and the units for the IBR, including refining the green crude to produce one or more fuel products, are all within close proximity or adjacent to each other.

The developed algae strain is grown in open ponds, in which the algae, water and nutrients circulate. As a result of the mixing, algae are circulated back up to the surface on a regular frequency. The ponds are shallow and operated continuously, such that water and nutrients are constantly fed to the pond, while algae-containing water is constantly removed at the other end.

The size of these ponds is measured in terms of surface area and productivity is measured in terms of biomass produced per day per unit of available surface area. The demonstration facility has approximately 300 acres of pond surface area, while the commercial facility has approximately 20,400 acres of pond surface area. The energy requirements for the production unit, comprising an input and production module, is shown in Table 2. The input module comprises the inputting of water and nutrients into the pond, whereas the production module comprises maintenance of the pond, such as keeping the flow of the water and keeping the algae suspended.

TABLE 2
Energy Requirements for Production Unit.

Facility:

| | Demonstration | Commercial |
|---|---|---|
| Input Module | 0.26 MW | 18 MW |
| Production Module | 0.15 MW | 11 MW |

### Processing Unit: Harvesting and Extracting

### Harvesting

Algae from the ponds is mixed with a flocculent and pumped to a settling tank to begin the dewatering process. The flocculent promotes algae settling. Following settling, algae at two percent solids is pumped to a process tank with membranes, such as GE Zeeweed membranes, submerged into the process liquid. Water permeates through the membranes and algae become more concentrated on the outside of the membranes. The Zeeweed membranes operate under a slight vacuum induced by the permeate pump, which pumps away water that flows through the membrane. The concentration of solids in the process tank is controlled to roughly five percent by the rate at which retentate is pumped away. Compressed air is fed to the bottom of the membrane module to prevent solids from accumulating on the outside surface of the membranes. The air also provides agitation that keeps solids suspended in the process tank. Permeate water is also periodically pumped in reverse (from the inside to the outside of the membrane) to remove any particles that may be lodged in the membrane interstices.

Following membrane separation, concentrated algae is pumped to a disc stack centrifuge to further separate water. The centrifuge decreases water content from approximately 95% to 90%. Water removed by the membrane and centrifuge steps is collected and returned to the ponds to capture the nutrients in the water. Concentrated algae are fed from the centrifuge to the extraction process. The energy requirement for harvesting is depicted in Table 3.

TABLE 3
Energy Requirements for Harvesting.

Facility:

| | Demonstration | Commercial |
|---|---|---|
| Harvest Module | 0.67 MW | 46 MW |

### Extracting

Algae oil is extracted from algae solids by mixing with hexane and a conditioning agent in a high-shear reactor. Hexane is recovered from the extracted oil and the solids left over after the extraction.

Algae oil contained within the algal solids is separated from biomass using high-shear contact with hexane and a conditioning agent. The oil dissolves into hexane, or other similar solvents, forming a solution called miscella. Water and cellular solids do not dissolve, and is collected separately from the miscella. The immiscibility of water and hexane is used to produce the desired separation. Following high-shear mixing, the algae/hexane/water mixture is sent to a decanter where it separates into two distinct liquids: a lighter hexane and oil phase (miscella), and a heavier water and spent solids phase.

Miscella from the decanter is fed to a distillation process where algae oil is separated from the solvent. This allows recovery and reuse of the solvent, and purifies the oil to a point where it is ready for downstream processing. Distillation takes advantage of the difference in boiling points of the solvent and oil to separate the two components.

Solids in the water phase are concentrated using a centrifuge or other mechanical concentration equipment. The water removed from the solids is recycled back to the ponds, while the solids, with some residual water, are fed to the solids handling section. The energy requirement for extracting is depicted in Table 4.

TABLE 4
Energy Requirements for Extracting.

| | Facility: | |
|---|---|---|
| | Demonstration | Commercial |
| Extraction Module | 11.2 MM Btu/hr; 0.5 MW | 764 MM Btu/hr; |
| | | 34 MW |

### Refining Unit

The finished oil, or green crude, is transported to a refinery. The refinery converts triglycerides from bio-renewable feeds such as fats, greases, and algae oils into a mixture of liquid hydrocarbon fuels, primarily green diesel and green jet, a high quality synthetic paraffinic kerosene (SPK). The refinery can be run in two different modes: a Mixed Mode, where output is a mixture of green diesel and green jet, and a Diesel Mode, where output is primarily green diesel.

During refining, the fatty acids and glycerides are converted to SPK in three steps. First, raw feedstocks are treated to remove catalyst contaminants and water. In the second step, fatty acid chains are transformed into n-paraffins in a hydrotreater. The example of oleic acid conversion to n-octadecane via the hydrogenation and deoxygenation reactions in the hydrotreater as shown in equation 1: C₁₇H₃₃COOH+4H₂->C₁₈H₃₈+2H₂O (1)

For most bio-oils, fats, and greases, the hydrotreater liquid product is a mainly C₁₅-C₁₈ n-paraffin composition. In the third step of the process, these long straight-chain paraffins are hydrocracked into shorter branched paraffins. The hydrocracked products fall mainly in the kerosene boiling range.

SPK meets or exceeds all jet fuel fit-for-purpose specifications except density. The high hydrogen-to-carbon ratio of SPK, which gives its excellent thermal stability and low participate emission attributes, means a lower density hydrocarbon composition: 760-770 kg/m³ compared to the minimum ASTM specification value of 775 kg/m³. However, this is not an issue with 50/50 blends of petroleum jet fuel and SPK.

The process requires hydrogen, which can be produced on-site using methane reforming, or can be provided by co-locating the facility at an existing refinery.

The commercial scale output has an approximately 5,000 bpd throughput, while the demonstration scale has an output of approximately 106 bpd of algae oil feedstock. The energy requirements for the refining process that can run in two modes is shown in Table 5.

TABLE 5
Energy Requirements for Refining Unit.¹

Facility:

| | IABR | Commercial |
|---|---|---|
| Dual Fuels Refining Unit | 0.1 MW | 10 MW |

¹The power value is an average over the time required to produce the algae oil feedstock. The refinery draws 5 MW of power during operation at 5,000 bpd throughput.

### Residual Solids Processing Unit

A residual solids processing unit is used to process the dried biomass produced from the extraction. Dried biomass from the extraction process is fed to an anaerobic digester, where the biomass is converted to a gas stream containing methane. The gas stream and residual nutrients are recycled to the production unit, or pond, for reuse. Dried biomass left over after digestion could be sold as animal feed. The energy requirements for the residual solids processing unit is shown in Table 6.

TABLE 6
Energy Requirements for Residual Solids Module.

| | Facility: | |
|---|---|---|
| | Demonstration | Commercial |
| Residual Solids Module | −5.4 MM Btu/hr; 0.03 MW | −368 MM Btu/hr; |
| | | 2 MW |

### Example 5

### IBR Using Dry Extraction Process

In this example, a demonstration facility and a second, larger facility, commercial scale, are built. The parameters of the facilities are shown in Table 7. Both facilities utilize substantially identical processes to convert oils extracted from algae into liquid transportation fuels.

TABLE 7
Design Parameters for Demonstration and Commercial Scale Facilities.

| | Facility: | |
|---|---|---|
| | Demonstration Scale | Commercial Scale |
| Algae Pond Acreage: | 300 | 20,400 |
| CO2 Capture (MT/day): | 56 | 4,878 |
| CO2 Utilization: | 60% | 90% |
| Extractable Liquid Fraction: | 50% | 60% |
| Refinable Liquid Fraction: | 85% | 90% |
| Refined Oil (bbl/day)¹ | 91 | 10,000 |

| Refinery Output Mode:² | Mixed | Diesel | Mixed | Diesel |
|---|---|---|---|---|
| Green Diesel (bbl/day): | 52 | 82 | 5,000 | 9,000 |
| Green Jet (bbl/day): | 32 | 0 | 4,300 | 0 |

¹Values are for mean productivity of algae ponds.
²Refining process to generate fuels can be run in two different modes: 1) Mixed Mode, where output is a mixture of green jet and green diesel, and 2) Diesel Mode, where output is primarily green diesel.

### Production Unit

One or more algal strains is developed and grown from a production unit as described in Example 4.

### Processing Unit Harvesting and Extracting

Harvesting

As described in Example 4, algae from the ponds is mixed with a flocculent and pumped to a settling tank to begin the dewatering process. The flocculent promotes algae settling. Following settling, algae at two percent solids is pumped to a process tank with GE Zeeweed membranes submerged into the process liquid. Water permeates through the membranes and algae become more concentrated on the outside of the membranes. The Zeeweed membranes operate under a slight vacuum induced by the permeate pump, which pumps away water that flows through the membrane. The concentration of solids in the process tank is controlled to roughly five percent by the rate at which retentate is pumped away. Compressed air is fed to the bottom of the membrane module to prevent solids from accumulating on the outside surface of the membranes. The air also provides agitation that keeps solids suspended in the process tank. Permeate water is also periodically pumped in reverse (from the inside to the outside of the membrane) to remove any particles that may be lodged in the membrane interstices.

Following membrane separation, concentrated algae is pumped to a disc stack centrifuge to further separate water. The centrifuge decreases water content from approximately 95% to 80%. Water removed by the membrane and centrifuge steps is collected and returned to the ponds to capture the nutrients in the water.

In this example, after growth and harvesting of the algae, the algae are dried prior to oil extraction. The concentrated algae are then fed from the centrifuge to a milling flash dryer. Because the algae from the centrifuge resembles a paste and will not dry easily in a flash dryer, dried algae is backmixed with fresh feed to produce a crumbly, free flowing material prior to introducing it into the drying chamber. Velocities within the torus of the flash dryer are sufficiently high so that the larger more dense particles remain to the outside radius of the dryer. The dryer exhaust is taken from the inside radius, thereby recycling the larger, still wet, material back to the milling area for further de-agglomeration and drying. Dryer exhaust is conveyed through a fan, which maintains the flash dryer at a slight negative pressure, before entering a bag collector. Dried algal solids collected in the bag collector is discharged to a conveyor that then feeds dry product to the backmixer to mix with the incoming wet feed material and conveys dry product to the oil extraction process. The energy requirement for harvesting is depicted in Table 8.

TABLE 8
Energy Requirements for Harvesting.

| | Facility: | |
|---|---|---|
| | Demonstration | Commercial |
| Harvest Module | 11.1 MM Btu/hr; 1.6 MW | 756 MM Btu/hr; |
| | | 109 MW |

### Extracting

Algae oil contained within the dried algal solids is separated from the dry biomass using counter-current contact with hexane. Oil dissolves into hexane, or other similar solvents, forming a solution called miscella. Cellular solids do not dissolve, and can be collected separately from the miscella. Most extractors utilize a conveyor system to draw the solids through the solvent solution, ensuring that the material is completely surrounded by miscella at all times. Solvent is usually pumped in the opposite direction of the conveyor. This countercurrent arrangement allows the extracted material to be discharged from one end of the machine while concentrated miscella (solvent and extractable) is taken from the other end. The solvent selected for extraction is less dense than the solids so that the powdery material left over after all the oil is extracted stays on the conveyor, and does not float on the surface of the miscella as it is collected. The concentrated miscella discharges from the extractor through a hydroclone, which scrubs fine particles from the oil/solvent mix before being pumped to the distillation system.

Algae's low density can make this separation more complicated than it is for some traditional vegetable oils. Traditional conveyors utilize a wire mesh surface. For the algal powder, the screen is very fine and significantly reduces solvent flow. The screen drains extremely slowly as well and minimizes separation efficiency, so a solid conveyor is utilized.

Miscella from the extractor is fed to a distillation process where algae oil is separated from the solvent. This allows recovery and reuse of the hexane solvent, and purifies the oil to a point where it is ready for downstream processing. Distillation takes advantage of the difference in boiling points of the solvent and oil to separate the two components.

Material from the solvent extractor contains between 20 to 40 percent solvent by weight. The material is desolventized, then dried and cooled before it is fed to the anaerobic digester. This process is accomplished in a desolventiser-toaster, which consists of a vertical stack of several cylindrical gas-tight pans, each having a steam-heated bottom.

Desolventizers generally have three sections: a pre-desolventizing section, a desolventizing section, and a toasting and stripping section. In the pre-desolventizing section, hexane is evaporated by indirect heating via heated trays. Solids continue to the desolventizing section, where most of the hexane is evaporated by condensing live steam. In the toasting and stripping section a combination of indirect and live steam is used to strip the remaining hexane while at the same time toasting the meal.

The solvent laden material enters the top of the desolventizer-toaster (DT) and land on the steam heated pre-desolventizing tray(s) where it is evenly distributed by a sweep arm. The material flows from one tray to the next through tray openings. As it rises up through the meal, the steam provides specific heat and a carrier gas to strip final traces of solvent from the material. The amount of live steam that is condensed is directly proportional to the amount of solvent in the material, one kg of condensing water vapor evaporating between 6 and 7 kg of hexane. The energy requirements for extracting is shown in Table 9.

TABLE 9
Energy Requirements for Extraction Module.

| | Facility: | |
|---|---|---|
| | Demonstration | Commercial |
| Extraction Module | 2.8 MM Btu/hr; 0.35 MW | 191 MM Btu/hr; |
| | | 24 MW |

### Refining Unit

The finished oil, or green crude, is transported to a refinery, as described in Example 4. The commercial scale refinery operates at approximately 5,000 bpd throughput, while the demonstration scale facility produces approximately 91 bpd of algae oil feedstock. The energy requirements are as described in Table 5.

Residual Solids Processing Unit

A residual solids processing unit, as described in Example 4, is used to process the dried biomass produced from the desolventizing. Dried biomass from desolventizing is fed to a residual solids processing unit, an anaerobic digester, where the biomass is converted to a gas stream containing methane. The energy requirements are as described in Table 6.

What is claimed is:

1. An integrated biorefinery (IBR) capable of producing jet fuel, diesel fuel, and gasoline from a single biofeedstock, comprising:
an open pond production unit of at least 350 acres for growing an aquatic non-vascular photosynthetic organism that produces an oil composition;
a processing unit for extracting said oil composition from said organism;
a refining unit for refining said oil composition to produce jet fuel, diesel fuel, gasoline or some combination thereof, wherein said refinery performs one or more process of cracking, transesterification, hydroprocessing, and isomerization of said oil composition;
a waste processing unit for processing residual matter from said processing unit; and
a conduit for delivering at least one product from said waste processing unit to said production unit for use in growth or maintenance of said organism.

2. The IBR of claim 1, wherein said organism is an alga.

3. The IBR of claim 1, wherein said processing unit further performs one or more processing of degumming, bleaching and deodorizing.

4. The IBR of claim 1, wherein said organism is grown in brackish water.

5. The IBR of claim 1, wherein said production unit uses a supplemental source of CO₂ to grow said organism.

6. The IBR of claim 5, wherein said supplemental source of CO₂ is obtained from flue gas.

7. The IBR of claim 1, wherein said organism is a genetically modified organism.

8. The IBR of claim 1, wherein said hydroprocessing is at least one of hydrotreating, hydrocracking and hydroisomerization.

9. The IBR of claim 8, wherein said hydrotreating is at least one of hydrodenitrogenation (HDN), hydrodeoxygenation (HDO) and hydrodemetallization (HDM).

10. The IBR of claim 1, wherein said cracking is at least one of thermal cracking, fluid catalytic cracking, thermal catalytic cracking, catalytic cracking, steam cracking, and hydrocracking.

11. The IBR of claim 1, wherein said refining unit is located at the same site as said production unit.

12. The IBR of claim 1, wherein said refining unit at a different location from said production unit.

13. The IBR of claim 12, wherein said refining unit is within 5 miles 10 miles, 25 miles, 50 miles, 75 miles, 100 miles, 250 miles or 500 miles of said production unit.

14. The IBR of claim 1, wherein said production unit is at least 500, 750, 1000, 2500, 5000, 10000, 20000 or 50000 acres.

15. The IBR of claim 1, wherein said waste processing unit comprises an anaerobic digester, an aerobic digester or both.

16. The IBR of claim 1, wherein said waste processing unit produces hydrogen, CO₂, minerals or some combination thereof.

17. The IBR of claim 1, wherein said waste processing unit produces power for use in at least one of said waste processing unit, said production unit, said processing unit and said refining unit.

18. The IBR of claim 17, wherein said IBR is energy self-sufficient.

19. The IBR of claim 6, wherein said flue gas is produced by combustion of fossil fuels.

20. The IBR of claim 19, wherein said jet fuel, diesel fuel or gasoline is at least 80% by weight of hydrogen and carbon, and wherein the carbon in said fuel product has a δ¹³C distribution of less than −40‰.
